SEQUENCE LISTING (40 sequences)

SEQ ID NO: 1 - DNA, 22 bp, Artificial Sequence
  Synthetic
gaatgcagactcagtggagaag

SEQ ID NO: 2 - DNA, 22 bp, Artificial Sequence
  Synthetic
cactgccatatccagaggaaac

SEQ ID NO: 3 - DNA, 20 bp, Artificial Sequence
  Synthetic
caggagctgatgaacgtcaa

SEQ ID NO: 4 - DNA, 20 bp, Artificial Sequence
  Synthetic
catgttctgcatcccagact

SEQ ID NO: 5 - DNA, 20 bp, Artificial Sequence
  Synthetic
gatgccgaggattggttctt

SEQ ID NO: 6 - DNA, 21 bp, Artificial Sequence
  Synthetic
gatctcactcttgccactctg

SEQ ID NO: 7 - DNA, 22 bp, Artificial Sequence
  Synthetic
gacctggactccatgagaaatc

SEQ ID NO: 8 - DNA, 20 bp, Artificial Sequence
  Synthetic
gttgagctgctccatctgta

SEQ ID NO: 9 - DNA, 21 bp, Artificial Sequence
  Synthetic
ccgaaagagaaagcgaaccag

SEQ ID NO: 10 - DNA, 21 bp, Artificial Sequence
  Synthetic
atgtggctgatctgctgcagt

SEQ ID NO: 11 - DNA, 19 bp, Artificial Sequence
  Synthetic
ccatgaccagctcgcagac

SEQ ID NO: 12 - DNA, 19 bp, Artificial Sequence
  Synthetic
ggacttgaccaccgaaccc

SEQ ID NO: 13 - DNA, 19 bp, Artificial Sequence
  Synthetic
gtggaccgcacggaatttg

SEQ ID NO: 14 - DNA, 20 bp, Artificial Sequence
  Synthetic
ggagattcacaccggagtca

SEQ ID NO: 15 - DNA, 18 bp, Artificial Sequence
  Synthetic
gaaaaccagcagctccag

SEQ ID NO: 16 - DNA, 18 bp, Artificial Sequence
  Synthetic
gcgctcgtgtaggccgtg

SEQ ID NO: 17 - DNA, 20 bp, Artificial Sequence
  Synthetic
aatgttggaggtctgcttgg

SEQ ID NO: 18 - DNA, 20 bp, Artificial Sequence
  Synthetic
cggtcagggcttcaatttcg

SEQ ID NO: 19 - DNA, 21 bp, Artificial Sequence
  Synthetic
gacggagcactttccttactt

SEQ ID NO: 20 - DNA, 22 bp, Artificial Sequence
  Synthetic
cctgatttgttctccaccttct

SEQ ID NO: 21 - DNA, 19 bp, Artificial Sequence
  Synthetic
gtacggtcaggcaccaaat

SEQ ID NO: 22 - DNA, 22 bp, Artificial Sequence
  Synthetic
cagcactgtaggtcatgtaagg

SEQ ID NO: 23 - DNA, 21 bp, Artificial Sequence
  Synthetic
ggctcagacgaactcctacta

SEQ ID NO: 24 - DNA, 23 bp, Artificial Sequence
  Synthetic
gaggatcctaaagcctgtgttac

SEQ ID NO: 25 - DNA, 21 bp, Artificial Sequence
  Synthetic
tgcctcaacccaatcctttat

SEQ ID NO: 26 - DNA, 22 bp, Artificial Sequence
  Synthetic
ccacactttgtctctgtcttct

SEQ ID NO: 27 - DNA, 22 bp, Artificial Sequence
  Synthetic
atggacagaagtggtggataag

SEQ ID NO: 28 - DNA, 21 bp, Artificial Sequence
  Synthetic
cctctccaaatgtcacaggaa

SEQ ID NO: 29 - DNA, 20 bp, Artificial Sequence
  Synthetic
gtctcagcttcagcgtcttt

SEQ ID NO: 30 - DNA, 22 bp, Artificial Sequence
  Synthetic
caccatccaaagggtgtctatc

SEQ ID NO: 31 - DNA, 21 bp, Artificial Sequence
  Synthetic
ccacctcttgtcagtgctcgg

SEQ ID NO: 32 - DNA, 22 bp, Artificial Sequence
  Synthetic
gggtttaatttccaggcacatg

SEQ ID NO: 33 - DNA, 21 bp, Artificial Sequence
  Synthetic
cagcaaggagaggatgaacac

SEQ ID NO: 34 - DNA, 24 bp, Artificial Sequence
  Synthetic
gggataggttcaacgaatggatag

SEQ ID NO: 35 - DNA, 19 bp, Artificial Sequence
  Synthetic
aagcaatgcagcagaacgc

SEQ ID NO: 36 - DNA, 21 bp, Artificial Sequence
  Synthetic
gtaaagccattaaagcagaag

SEQ ID NO: 37 - DNA, 22 bp, Artificial Sequence
  Synthetic
gaacaccatggagaccatagac

SEQ ID NO: 38 - DNA, 19 bp, Artificial Sequence
  Synthetic
aggagtgcctgctcatttc

SEQ ID NO: 39 - DNA, 20 bp, Artificial Sequence
  Synthetic
tgcaccaccaactgcttagc

SEQ ID NO: 40 - DNA, 21 bp, Artificial Sequence
  Synthetic
ggcatggactgtggtcatgag

(19) United States
(12) Patent Application Publication
(10) Pub. No.: US 2023/0002727 A1
(43) Pub. Date: Jan. 5, 2023

(54) THYMUS ORGANOIDS BIOENGINEERED FROM HUMAN PLURIPOTENT STEM CELLS

(71) Applicants: Allegheny Singer Research Institute, Pittsburgh, PA (US); University of Pittsburgh - of the Commonwealth System of Higher Education, Pittsburgh, PA (US)

(72) Inventors: Yong Fan, Pittsburgh, PA (US); Ann Zeleniak, Pittsburgh, PA (US); Massimo Trucco, Pittsburgh, PA (US); Ipsita Banerjee, Pittsburgh, PA (US)

(21) Appl. No.: 17/756,398

(22) PCT Filed: Nov. 25, 2020
(86) PCT No.: PCT/US2020/062180

§ 371 Date: May 24, 2022

Related U.S. Application Data
(60) Provisional application No. 62/939,918, filed on Nov. 25, 2019.

(51) Int. Cl. C12N 5/078; A01K 67/027; A61K 35/26; C12N 5/00; C12N 5/0789; C12N 5/071

(52) U.S. Cl. CPC C12N 5/065; A01K 67/0278; A61K 35/26; C12N 5/0012; C12N 5/0647; C12N 5/0697; A01K 2207/12; A01K 2217/15; A01K 2227/105; C12N 2501/125; C12N 2501/145; C12N 2501/15; C12N 2501/155; C12N 2501/16; C12N 2506/45; C12N 2513/00; C12N 2533/74; C12N 2533/90

(57) ABSTRACT

This document relates to bioengineering and involves bioengineered thymus organoids and related humanized animal models. The thymus organoids and animal models have various commercial and clinical uses, including generating humanized antibodies, making antigen-specific human T cells, inducing transplantation tolerance, rejuvenating thymus functions, and modeling human diseases.

### RELATED APPLICATION AND CLAIM OF PRIORITY

This application claims priority to U.S. Provisional Application No. 62/939,918 filed on Nov. 25, 2019. The content of the application is incorporated herein by reference in its entirety.

### GOVERNMENT INTERESTS

This invention was made with government support under Grant Nos. R21 AI126335 and R01 AI123392 awarded by the National Institutes of Health and under Grant No. 1804728 awarded by the National Science Foundation. The government has certain rights in the invention.

### FIELD OF THE INVENTION

This document relates to bioengineering and involves bioengineered thymus organoids, related humanized animal models, and related uses.

### BACKGROUND

The thymus, a pivotal immune organ in the adaptive immune system, is responsible for generating a diverse repertoire of T-cells that can effectively react to invading pathogens, while maintaining immune self-tolerance. Numerous factors varying from aging, chemotherapy, radiation exposure, virus infection and inflammation contribute to thymus involution, a phenomenon manifested as loss of thymus cellularity, increased stromal fibrosis and diminished naïve T-cell output. Impaired immune surveillance consequent to thymic dysfunction leads to diseases ranging from autoimmunity to immunodeficiency and malignancy. There is need to restore or improve impaired immune functions due to thymus defects.

One of the major hurdles in translating experimental findings from animal models, such as mouse models, into clinical applicable therapies is the existence of species-specific differences between the mouse and human. Humanized mice, in which human immune cells are engrafted and populated in the immune deficient mice, provide a powerful tool to study the development and responses of human immune system in vivo. One of the major obstacles of recapitulating the human immune system in mice is the defective development of human T cells, due to the limitation of mouse thymic microenvironments in supporting human T cell development and selection. While co-transplantation of fetal liver and thymus in the human bone marrow-liver-thymus (BLT) mouse model can support robust T cell development, the frequent incidence of graft-versus-host disease (GVHD) result in short life spans of the hosts, rendering them prohibitory to be used for long-term studies. Potential ethical issues, as well as the lack of human leukocyte antigen (HLA) selection in fetal tissues, further limit the use of the BLT mice in modeling human immune responses under various pharmacological, physiological and pathological conditions.

### SUMMARY

This document addresses the needs mentioned above in a number of aspects.

In one aspect, the document provides a method for making a bioengineered thymus organoid. The method comprises obtaining a cell population comprising human thymic epithelial progenitor cells (TEPCs) or human thymic epithelial cells (TECs) or both; combining the cell population with human hematopoietic stem cells (HSCs) in a defined ratio to form a combination; seeding the combination into an extracellular matrix of a de-cellularized thymus scaffold to generate a thymus construct, and culturing the thymus construct under conditions permitting cellular attachment onto the extracellular matrix thereby making the bioengineered thymus organoid. The TEPCs, the TECs, or the HSCs can be derived from a donor human individual. The HSCs may comprise human CD34+ hematopoietic stem cells. The de-cellularized thymus scaffold can be from a human subject or a non-human donor animal. As mentioned below, various suitable animals can be used as the donor animal. Preferred examples include non-human mammals. The ratio of the TEPCs or TECs to the HSCs can range from 100:1 to 1:100, such as about 10:1 to 1:10, or about 1:10, 1:1, 2:1, or 5:1.

In some embodiments, the cell population can be obtained by a process comprising encapsulating human pluripotent stem cells (hPSCs) in a suspension medium that separates the hPSCs into single cells; culturing the hPSCs in a growth medium to increase the number thereof or to obtain progeny cells thereof without differentiation; differentiating the hPSCs or progeny cells to generate TEPCs or TECs in an encapsulation medium, and freeing the TEPCs or TECs from the encapsulation medium. Examples of the hPSCs can include human induced pluripotent stem cells (hiPSCs) or human embryonic stem cells (hESCs).

In some examples, the thymus construct can be placed into a flow cell in vitro with a continuous feed of a medium or nutrients and human cells to produce human immune cells. The thymus construct may comprise immune cells, such as B-cells and T-cells. The B-cells may be specific to a particular antigen or may produce antigen-specific antibodies. In one embodiment, the T cells may be transduced with a viral vector encoding a chimeric antigen receptor (CAR). For instance, the viral vector can be added to the flow cell to transduce the T cells. The thymus construct can be used for evaluating a drug candidate. In that case, the thymus construct can contacted with a drug candidate to test the impact of the drug candidate on immune cell development.

In some other examples, the thymus construct can be surgically transplanted to a host animal. The host animal can be a preconditioned humanized immune-deficient animal, such as a preconditioned humanized immune-deficient pig, rat or mouse. The thymus construct may be placed in various suitable locations in the host animal, and preferably, at an anatomic site with rich blood vessel network, such as under the kidney capsule, in the thoracic area in the neck, or in the axillary region. The resulting host animal can be provided HSCs and produces human immune cells. For example, the host animal can be a non-human animal (e.g., a mouse, such as those developed by ABGENIX or MEDAREX) engineered to express human VDJ antibody sequences. Such an animal can produce increased quantities of fully human Immunoglobulin G and antibodies. The resulting host animal can also be used for evaluating a drug candidate. In that case, the host animal can be administered with a drug candidate to test the impact of the drug candidate on immune cell development. For instance, the TEPCs, the TECs, or the HSCs can be from a donor individual to test the impact of the drug candidate on the donor individual. The host animal can also be transplanted with cells or a tissue from the donor individual. Examples of the cells or the tissue may include cancer cells. In that case, the effect of the drug candidate on the cells or tissue from the donor individual can be evaluated.

In the methods described above, the donor animal or the host animal can be a vertebrate including a non-mammal, such as bird, amphibian, reptile, fish (e.g., zebra fish) or other jawed vertebrates, or a non-human mammal. Examples of the non-human mammal include one selected from the group consisting of cow, pig, camel, llama, horse, goat, rabbit, sheep, hamsters, guinea pig, cat, dog, rat, mouse, and a non-human primate.

In a second aspect, the document provides a bioengineered thymus organoid, comprising (i) human TEPCs (hTEPCs), human TECs (hTECs), or human HSCs (hHSCs) and (ii) a thymus scaffold that has been de-cellularized and comprises an extracellular matrix, wherein the hTEPCS, hTECs, or hHSCs attach to the extracellular matrix. Examples of the bioengineered thymus organoid includes a bioengineered thymus organoid prepared according to the methods described above. The bioengineered thymus organoid may comprise immune cells. The thymus scaffold can be heterologous or allogeneic to the hTEPCs, hTECs, or hHSCs. 29. In some examples, the hTEPCs or hTECs may be derived from hPSCs. The bioengineered thymus organoid can be in vitro or in vivo. Accordingly, within the scope of this document is a non-human animal comprising the bioengineered thymus organoid describe above. The non-human animal may be a mammal selected from the group consisting of cow, pig, camel, llama, horse, goat, rabbit, sheep, hamsters, guinea pig, cat, dog, rat, mouse, and a non-human primate.

In a further aspect, the document provides a method for evaluating of a drug candidate. The method comprises (a) contacting a drug candidate with the bioengineered thymus organoid described above and (b) detecting the impact of the drug candidate on development of cells that are in the bioengineered thymus organoid or emigrate therefrom. The method can be carried out in vitro or in vivo in a host animal. In one example, the bioengineered thymus organoid may be implanted in a host animal and the drug candidate is administered to the host animal.

For evaluating of a drug candidate as described herein, the drug candidate can be one selected from the group consisting of a small molecule, a nucleic acid, a peptide, a polypeptide, an antibody, and an antibody fragment.

In yet another aspect, the document provides a method of preparing thymic emigrant cells. The method includes (a) introducing progenitor cells into the bioengineered thymus organoid described above or the non-human animal described above, (b) maintaining the bioengineered thymus organoid or the non-human animal under conditions permitting differentiation of the progenitor cells to generate progeny cells thereof; (c) egressing the progeny cells from the bioengineered thymus organoid to generate thymic emigrant cells, and (d) isolating the thymic emigrant cells. Accordingly, within the scope of this document are thymic emigrant cells prepared according to the method. The thymic emigrant cells may comprise one or more transgenes encoding an antigen receptor, such as a chimeric antigen receptor (CAR).

The thymic emigrant cells can be included in a pharmaceutical composition comprising the thymic emigrant cells and a pharmaceutically acceptable carrier. The thymic emigrant cells or the pharmaceutical composition can be used in a method for improving the immune function of a subject in need thereof. Accordingly, this document provides a method for improving the immune function of the subject. The method includes (a) administering to the subject an effective amount of the thymic emigrant cells, or (b) transplanting to the subject the bioengineered thymus organoid described above. In some examples, the subject may have a condition selected from the group consisting of cancer, autoimmune disorder, and infection.

### BRIEF DESCRIPTION OF THE DRAWINGS

FIGS. 1A, and 1B are diagrams showing differentiation of human iPSCs into TEPCs in 3-D alginate hydrogel capsule. a. Size distribution of iPSC aggregates at different stages of TEPC differentiation. The open shapes in the left panel indicate the size of individual aggregates in the capsule. Solid lines show the overall size distribution of TEPCs aggregates at each stage (left panel), which is also presented as box plot in the right panel. # indicates no significance between means p<0.05 (one way ANOVA and post-hoc Tukey test). b. Representative histogram of flow cytometry analysis of EpCAM+dissociated TEPCs.

FIGS. 2A, 2B, 2C, 2D, 2E, 2F, 2G, and 2H are diagrams showing tissue-engineering functional thymus organoids from iPSC-derived TECs. a. RT-qPCR analysis of expression of genes critical for antigen-presentation function. Shown are results of triplicates from at least three independent experiments. *p<0.05; **p<0.01; ***p<0.005. b. Representative FCM graphs showing the development of T cells within the thymus organoids (iPSC organoids), or among cells flowing out of the organoids (iPSC Efflux). L/D, live/dead staining, in which dead cells are stained positively. c-h. Thymus organoids were constructed with iPSC-TECs and transplanted underneath the kidney capsules of athymic B6 nude mice. Representative FCM graphs showing the presence of CD3+CD45+ cells (c and d), CD3+CD4+ and CD3+CD8+ T cells (e), γδ T cells (f) in spleens and lymph nodes of thymus organoid engrafted mice 18-32 weeks post-transplantation. Nu, B6.nude mice controls; Thy, thymus organoid-engrafted B6.nude mice. g. Activation status of CD4+(left panel) and CD8+(right panel) T cells in lymph nodes. h. Mixed lymphocyte reaction (MLR). Representative histograms showing the proliferation responses of CD4+(left panels) and CD8+(right panels) T cells isolated from the thymus organoid-engrafted B6.nude mice, when challenged with mitomycin C-treated syngeneic (B6, top panels) or allogeneic (Balb/C, lower panels) splenocytes. Shown are representative results of triplicate from three independent experiments. *p<0.05; **p<0.01.

FIGS. 3A and 3B are diagrams showing generation of iPSC-derived TEC thymus organoid-engrafted hematopoietic humanized mice. a. Kaplan-Meier survival analysis of humanized mice post-transplantation. ****, p<0.001 (Logrank test). b. Percentages of hCD45+ cells in peripheral blood of four groups of humanized mice (G1-G4) at 12-week post-transplantation. *p<0.05; ****p<0.001 (Mann-Whitney test).

FIGS. 4A, 4B, 4C, and 4D are diagrams showing development of multiple hematopoietic lineages in hu.Thor mice. Cells were isolated from the bone marrows (BM) and spleens (SPL) of G1-G4 humanized mice (18-40 weeks post-transplantation) and analyzed with FCM for overall ratios of human cell chimerism (% of hCD45 in total CD45+ cells) (a-c.), and for the presence and distribution of various hematopoietic lineages (d). d. Representative pie chart showing the distribution of human hematopoietic lineages of control (G3) and hu.Thor (G4) mice. Shown are representative results from three independent experiments. *p<0.05; **p<0.01 (Mann-Whitney test).

FIGS. 5A, 5B, 5C, 5D, 5E and 5F are diagrams showing development of functional human T cell subsets in hu.Thor mice. a. Overall diversity of TCR Vβ gene family expression in hu. Thor mice (n=3). RNA was isolated from splenocytes of hu. Thor mice (red columns), and PBMC of healthy donors (blue column, n=3). Vβ gene expression was analyzed with NanoString T cell panel. Shown are percentages of total TCR Vβ gene expression for each Vβ family (mean±SEM). b-d. Bone marrow and spleens were harvested from hu.Thor mice (n=4) at 31 weeks post-transplantation and characterized with FCM for human T cell subsets. All cells were first gated on the hCD45+ populations, unless specified otherwise. b-c. Representative FCM graphs showing the development of CD45RA+CD45RO− naïve, and CD45RA−CD45RO+ memory CD4+ and CD8+ T cells (b), and subsets of CD4+T helper cells (c). d. Splenocytes harvested from hu. Thor mice were stimulated with PMA+ionomycin (lower panels) or medium+DMSO control (upper panels) and intracellularly stained with antibodies against hIL17A and hIFNγ (right panels). e. MLR experiments showing the proliferation responses of hCD45+CD3+ T cells of Y1 hu.Thor mice challenged with HLA-mismatched human cord blood samples (hCBs 6 and 18 in Table 3). Shown are representative FCM graphs of three repeats from two independent experiments. f. Overall diversity of TCR Vα gene families in hu.Thor mice. RNA was isolated from splenocytes of hu.Thor mice (n=3, red columns), and PBMC of healthy donors (blue column, n=3). Vα gene expression was analyzed with NanoString T cell panel. Shown are percentages of total TCR Vα gene expression for each Vα family (mean±SD).

FIGS. 6A and 6B are diagrams showing pathway analysis of genes associated with T cell function shows similar expression profiling between hu. Thor immune cells and PBMCs. Total RNA were isolated from splenocytes of hu.Thor (n=2) and hu.SRC mice (n=4), as well as PBMC of healthy donors (n=2). Expression of a panel of T cell related genes was examined with the nCounter direct digital detection technology. Pathway score analysis was performed, in which a score is calculated as the first principal component of the set of genes relevant to each specific pathway, to reflect its overall property. Summary plot of pathway score analysis shows similar trend of T cell-relevant pathways between hu. Thor and PBMC cells, in striking contrast to hu. SRC cells. Pathway score analysis of specific pathways relevant to TCR signaling, diversity, T-helper subset (a), as well as activation (b). Overall expression levels of genes relevant to T cell exhaustion.

FIG. 7 is a set of diagrams showing effective rejection of allogeneic iPSC-derived teratomas in hu. Thor mice on weight of tumors derived from the allogeneic CC1 line (left panel) and Y1 line (right panel).

FIGS. 8A and 8B are diagrams showing human T cells derived from iPSC-thymus organoids can modulate the humoral immune responses in hu.Thor mice. a. Generation of major human immunoglobulin classes in hu. Thor mice. Sera were harvested from hu.Thor mice at 16-18 weeks post-transplantation. Isotypes of human immunoglobulin classes were quantified with LUMINEX isotyping kit. Sera from untreated NSG and hu.SRC mice at similar post-transplant ages were used as controls. b. hu.Thor mice were intramuscularly injected with 50 μl of clinical grade diphtheria toxoid (DT) vaccine, with a booster injection after one week. Serum samples were harvested before immunization (pre-bleed, Pre), 1 week after immunization (Post), and four weeks after the initial immunization (Boost). DT-reactive IgG antibodies were measured with ELISA. Shown are fold increase of OD450 values in relative to Pre samples. n=3-5. *, p<0.05.

FIGS. 9A and 9B show differentiation of human hESCs into TEPCs in 3-D alginate hydrogel capsule and size distribution of iPSC aggregates at different stages of TEPC differentiation. a. The open shapes indicate the size of individual aggregates in the capsule. Solid lines show the overall size distribution of TEPCs aggregates at each stage. b. Box plot of size distribution is shown and # indicates no significance between means p<0.05 (one way ANOVA and post-hoc Tukey test).

FIG. 10 shows tissue-engineering functional thymus organoids from hESC-derived TECs. Human thymus organoids were constructed by co-injecting H1 hESC-TECs and CD34+ cord blood into decellularized murine thymus scaffolds. Thymus organoids were cultured in the top chambers of transwell culture system for 3 weeks. Representative FCM graphs showing the development of T cells within the thymus organoids (H1 organoids), or among cells flowing out of the organoids (H1Efflux). L/D, live/dead staining, in which dead cells are stained positively.

### DETAILED DESCRIPTION

This document relates to bioengineering and involves bioengineered thymus organoids and related humanized animal models. The thymus organoids and animal models (such as mouse models) have various commercial and clinical uses, including generating humanized antibodies, making antigen-specific human T cells, inducing transplantation tolerance, rejuvenating thymus functions, and modeling human diseases.

The thymus gland is the primary lymphoid organ responsible for the development of T-cells. It is organized into two morphologically and functionally distinct compartments, the cortex and the medulla, which house two distinctive populations of thymic epithelial cells (TECs): the cortical TECs (cTECs) and the medullary TECs (mTECs), respectively. Other populations of thymic stromal cells (TSCs) include thymic fibroblasts, endothelial cells, as well as macrophages and dendritic cells of hematopoietic origin. Together, this network of TSCs provides both homing signals for the immigration of common lymphocyte progenitors (CLPs) originated from the bone marrow (BM), and trophic factors necessary for the differentiation and maturation of thymocytes and T-lymphopoiesis.

T-lymphopoiesis is a well-coordinated process that involves continuous crosstalk between the developing thymocytes and the TECs. Early stages of T cell development (e.g. lineage specification, proliferation, TCR gene recombination, and positive selection) take place in the cortical region and are mediated mainly by cTECs. The resulting CD4+CD8+ double-positive (DP) cells express TCRs that can interact with the self-peptide presenting MHC (pMHC). Subsequently, DP cells are negatively selected in the medullary region, where cells expressing TCRs with high affinities to self-antigens are induced to undergo apoptosis by mTECs and APCs of hematopoietic origin, and differentiate into CD4+CD8− or CD4−CD8+ single-positive (SP) cells, before being released into circulation to be part of the diverse, but self-tolerant T-cell repertoire in the periphery.

The 3D organization of the thymic stroma is important for its function. Manipulation of the thymic stromal compartment, either in vitro or ex vivo, proves to be challenging. The bottleneck is mainly attributed to the unique architecture of the thymic stroma that is important for the survival and function of TECs. Unlike epithelial cells of other visceral organs typically forming in a 2D sheet-like structure, TECs are organized in a sponge-like, 3D network. TECs in 2D culture start to express markers of terminally differentiated, senescent epithelial cells, or even transdifferentiate into skin cells. Expression of key genes for the specification and proliferation of TECs (e.g. FoxN1, DLL-4, CLL-22 and Tbata) are also shown to be dependent on the 3D organization of the thymic stroma.

### Thymus Organoids and Preparation Methods

The document provides a bioengineered thymus organoid and a method of making the thymus organoid in vitro. An organoid is an in vitro, three-dimensional, miniature version of an organ. Thymus organoids produced by the method can mimic the physiology and function of a human thymus. A thymus organoid described herein may comprise, among others, thymic cells (e.g., TEPCs) derived from a human donor source and a scaffold derived from a different donor or a non-human animal. The thymus organoid can further comprise other cells (e.g., progenitor cells) from a human source, which can differentiate within the thymus organoid in vitro or in vivo in a host animal to produce cells useful for various purposes.

TEPCs

A thymus organoid described herein comprises, among others, thymic cells (e.g., TEPCs) derived from a human source. Various methods can be used to make the TEPCs. In certain embodiments, the method may comprise differentiating pluripotent stem cells into TEPCs in vitro. In this regard, the method may comprise culturing the pluripotent stem cells for a time and under conditions sufficient to differentiate the pluripotent stem cells into TEPCs. For example, the method may comprise culturing the pluripotent stem cells in different stages in the presence of members of the TGFβ superfamily (e.g., activin A), or a combination of Wnt family member 3A (Wnt3A), bone morphogenic protein 4 (BMP4), and fibroblast growth factor (FGF).

Various pluripotent stem cells may be used to generate TEPCs. In general, such pluripotent stem cells have the capacity to give rise to any of the three germ layers: endoderm, mesoderm, and ectoderm. Pluripotent stem cells may comprise, for example, stem cells, e.g., embryonic stem cells, nuclear transfer derived embryonic stem cells, induced pluripotent stem cells, etc. The pluripotent stem cells, e.g., iPSCs, may express any one or more of a variety of pluripotency-associated genes or markers. Pluripotency-associated genes or markers may include, but are not limited to, Oct-3/4, Sox2, Nanog, GDF3, REX1, FGF4, ESG1, DPPA2, DPPA4, hTERT, SSEA1, SSEA-3, SSEA-4, TRA-1-60, TRA-1-81, TRA-2-49/6E, and Nanog.

A number of protocols can be used for differentiating pluripotent stem cells (e.g., ESCs and iPSCs) into TEPCs. Although stem cell-derived TEPCs express markers of the thymic epithelium, further maturation in vivo (e.g., engraftment underneath the kidney capsules of athymic nude mice) can be used to gain the T-lymphopoiesis function. See, e.g., Parent, A. V. et al. Generation of functional thymic epithelium from human embryonic stem cells that supports host T cell development. Cell stem cell 13, 219-229, doi:10.1016/j.stem.2013.04.004 (2013), Sun, X. et al. Directed differentiation of human embryonic stem cells into thymic epithelial progenitor-like cells reconstitutes the thymic microenvironment in vivo. Cell stem cell 13, 230-236, doi:10.1016/j.stem.2013.06.014 (2013), and Chhatta, A. R. et al. De novo generation of a functional human thymus from induced pluripotent stem cells. J Allergy Clin Immunol 144, 1416-1419 e1417, doi:10.1016/j.jaci.2019.05.042 (2019). Yet, none of the previously generated hPSC-TEPCs have shown the ability to induce the differentiation of human HSCs into T cell lineages in vitro. Notably, traditional 2-D culture conditions were used in all these methods.

The method described in this document may comprise differentiating pluripotent stem cells in a 3D culture where the cells may further differentiate into TEPCs and/or TECs, including cTECs and mTECs. These cell types can be assessed using one, two, or more corresponding markers, including EpCAM1, CK5, CK8, CK17, CK18, OCT4, SOX17, SOX2, HOXA3, EYA1, PAX9, FOXN1, PRASS16, ACKR4, CD205, β5T, AIRE, and CSN2. As disclosed herein, the 3D culture may be more effective for facilitating thymus organoid development as compared to 2D culture, e.g., 2D culture of thymic stromal cells in monolayer cultures. Suitable 3D culture systems may include any 3D culture system, such as suspension in alginate capsule, hanging drop plates, and ultra-low attachment multiwell plates.

The 3-D configuration of TECs in the thymic microenvironment is important to maintain their thymic epithelial gene signature. For example, the expression of Tbata, a key transcription regulator to maintain TEC size and proliferation, drops drastically in 2-D TEC culture. Moreover, TEC progenitors isolated from mouse embryos display markers of skin keratinocytes in 2-D adherent culture. In contrast, TECs cultured as aggregates in biocompatible hydrogel can maintain their molecular properties and prolong their survival for up to 7 days in vitro. The data from this document show that the alginate hydrogel capsules used in the study might provide critical 3-D matrix support for the survival of iPSC-derived TEPCs.

For example, in comparison to 2-D TEPCs, the expression of cTEC-specific genes (e.g. PRSS16, ACKR4, CD205, and β5t) that are initially transcribed in the thymus glands of late stage embryos and/or neonates, was increased significantly in 3-D iPSC-derived TEPCs. Similar increase was detected in the expression of epithelial markers (e.g. CK-5, CK-8, CK-17 and CK-18). These findings suggest that 3-D TEPCs represented later stages of developing TECs than those of 2-D cultures. Exposure of 3-D iPSC-derived TEPCs to the ECM microenvironment of decellularized thymus scaffolds promoted further maturation of TECs in vitro, as suggested by the significant increase of expression of genes associated with antigen presentation (e.g. MHC II and CD74). Indeed, human thymus organoids constructed with 3-D iPSC-derived TECs successfully supported the differentiation of human CD34+HSCs into both DP and SP T cells in vitro, highlighting their thymopoiesis function. Accordingly, in an embodiment, the thymus organoid produced by the method may comprise high expression levels of one, two, more, or all of the above makers, in particular, CK-5, CK-8, CK-17, CK-18, EYA1, PAX9, FOXN1, PRASS16, ACKR4, CD205, β5T, AIRE, and CSN2.

Thymus Scaffold

As mentioned above, the unique architecture and microenvironment of the thymic stroma is important for the survival and function of TECs. The thymus organoid disclosed herein may contain a thymus scaffold, comprising an extracellular matrix from thymus, which provides the microenvironment for recolonization of TECs and related thymus function such as T-lymphopoiesis. In a preferred embodiment, the thymus scaffold is a de-cellularized thymus scaffold, such as a de-cellularized thymus tissue or organ or gland from a donor animal.

Various methods can be used to make the scaffold. One example uses a detergent-perfusion based method that allows the clearance of the cellular compartment of almost any organ of any scale, while retaining its ECM components largely intact. Shown below is an example protocol to remove all the cells in the thymus gland, while retaining its 3D ECM structure. In this protocol, de-cellularization is achieved with several rounds of freeze/thaw cycles to induce intracellular ice crystal formation, in conjunction with detergent treatment for cell lysis. The example protocol includes:

(1) Thymus glands are harvested from 3-24 weeks old mice, and placed in a Styrofoam box and frozen for 25 min at −80° C.

(2) Frozen thymic samples are later thawed in 30° C. water bath for 30 min.

(3) Steps (1) and (2) are repeated 1-2 times.

(4) Thymic samples are transferred to 12-well plates with 2 mL of 0.5% SDS solution and placed on a plate on a shaker at room temperature. The clearness of the thymic samples are monitored every hour and the 0.5% SDS is changed every 1.5-2 hours. This process is repeated 2-3 times. Larger thymus samples may need 1-2 more rounds of processing.

(5) 0.5% SDS is replaced with 0.1% SDS and samples are further decellularized overnight at 4° C. (cold room) on a shaker.

(6) Place remaining thymus matrix onto a cell strainer fitted to accommodate the sample.

(7) Perform 3×15-minute washes with 2 mL of ddH2O MgSO4, CaCl2 (5 mM), TRITON-X (1%). Transfer the remaining thymus matrix to a new well for each wash. Thymus samples will become more opaque for the first wash only. If the sample continues to become cloudier, it may be unfit for further use as a scaffold. Samples should remain mostly transparent.

(8) Perform 3×15-minute washes with 2 mL of PBS. Again, Transfer the thymus to a new well for each wash.

(9) Store thymus scaffolds in a round 96-well plate dry. Cover with a plate sticker and place in 4° C. (under refrigeration), or store the thymus scaffolds in 2 mL PBE+Pen-Strep. Place in 4° C. (under refrigeration).

To construct the thymus organoid, thymic cells such as TEPCs can be seeded or injected into the acellular thymic scaffolds and cultured in vitro. Survival of the cells can be assessed with techniques known in the art. As shown in the examples below, the cells can effectively colonize the ECM of the thymic scaffolds and remain alive for up to weeks. The molecular signature of thymus stroma is largely maintained in the thymus organoid, as demonstrated by RT-PCR analysis of TEC-specific genes.

The thymic organoid described herein has one or more of a variety of advantages. For example, human iPSC-derived thymic organoids produced by the method can produce thymic emigrant cells, such as cells of the T-cell lineage, which are useful for treating or preventing a condition in a mammal, e.g., cancer. Additionally, the method may provide an organoid that can mimic the positive selection processing that takes place in the thymus. The positive selection process refers to the ability of newly formed thymocytes to recognize and interact with MHC (major histocompatibility complex). During positive selection in the human thymus, only thymocytes that can bind to MHC will survive, migrate into the medulla, and differentiate into mature T cells.

In some embodiments, the thymic organoid allows one to produce thymic epithelial cells in a 3D organization in a way resembling the human thymus. In embodiments, the method may produce an organoid capable of differentiating T cells in vitro. In embodiments, the method and thymic organoid may provide a way to produce autologous T cells. For example, the document discloses methods of generating immune cells, such as T and NKT cells, for rare blood types for blood banking and the treatment of conditions or deficiencies, e.g., anemias and other cytopenias. In some embodiments, the method may generate T and NKT cells for patients with immunodeficiencies.

### Lymphopoiesis

The thymus organoid produced by the methods may be useful for generating various thymic emigrant cells including cells of the T cell lineage for adoptive cell therapy. Accordingly, an embodiment of this document provides a method of preparing thymic emigrant cells in vitro, in vivo, or ex vivo.

The method may comprise seeding or introducing suitable progenitor cells into the thymus organoid. For example, any progenitor cells which have the potential to develop into the T cell lineage can be migrated into the thymus organoid. Examples of suitable progenitor cells may include, but not limited to, primitive mesoderm cells, hematopoietic progenitors, pluripotent stem cell-derived cells, hematopoietic stem cells, T cell progenitors, double positive T cells, and immature T cell lineage cells. Various methods can be used to obtain the suitable progenitor cells. Shown below is an example protocol for obtaining CD34+ HSCs from umbilical cord blood samples:

(1) Umbilical cord blood samples (˜100 mL) are obtained from VITALANT. CD34+HSCs are isolated with MILTENYI CD34+ human hematopoietic stem cell magnetic beads, following manufacturer's protocol.

(2) CD34+HSCs are cryopreserved at 1×10⁵ to 2×10⁵ per tube in liquid nitrogen until use.

(3) CD34− cells are used to isolate genomic DNA, with QIAGEN blood/cell DNA isolation kit, or other methods. Genomic DNA is subjected to HLA testing, using HLA typing service. DNA from iPSC and hESC lines described elsewhere is subjected to HLA typing. In later steps, those CD34+HSCs that carry HLA molecules at matching the iPSCs and hESC lines are used for thymus reconstruction.

(4) Five to seven days prior to thymus organoid construction, CD34+HSCs are recovered and expanded in 6-well tissue culture dish, using a serum free hematopoietic stem cell culture and expansion medium (examples of which are available from Miltenyi Biotec or Stemcell Technologies).

Shown below is an example protocol for construction of thymus organoids containing CD34+HSCs for generating T cells:

(1) Construction of thymus organoids:
(1.1) Decapsulating the alginate capsules to retrieve the human TEPCs.
   (1.1.1) Spin down capsules to remove media for 5 min at 250 rcf.
   (1.2.1) Aspirate media supernatant by slightly tilting the tube to the side when the liquid level gets close to the capsules will help remove the media that is below the capsule surface.
   (1.3.1) Add approximately 7 mL of the 100 mM EDTA solution to the alginate capsules and place on the rocker/shaker for 5 minutes at room temperature. After 5 minutes, little white cell aggregates are visible in the solution.
   (1.4.1) Spin down solution for 5 minutes at 250 rcf to collect the cells. If there are visible residual alginate capsules, repeat steps 1.2.1 and 1.3.1.
   (1.5.1) Aspirate supernatant and resuspend the cells in the media used for seeding in the scaffold.
   (1.6.1) A small portion of the TEPCs can be used for quality controls. Specifically. About 500,000 to 1,000,000 cells can be stained with anti-EpCAM antibody (a specific marker for TECs). Antibodies against other epithelial cell markers, such as Krt18 can also be used). A good batch should have >60-70% of EpCAM+ cells.
(1.2) Construction of thymus organoids.
   (1.2.1) 1-3×10⁵ CD34+HSCs and 1-10×10⁵ TEPCs are mixed, centrifuged, aspirated, and resuspended in 20-50 μL of injection solution. The HSC:TEPC ratios used can vary from 1/10 to 10/1.
   (1.2.2) Load the cell mix into a 20 μL Hamilton syringe (with 33 gauge needle), or glass needles freshly pulled from micro-pipettes over the flame (if glass needles are used, the end of the glass needles will be connected to a rubber tubing, with a syringe at the other end).
   (1.2.3) Transfer thymus scaffolds to the cover of petri dish. Use forceps in one hand to stabilize the thymus scaffold. Pierce through the thymus scaffold membrane with the tip of the needle, and slowly inject about 20 μL of cells into one lobe. Repeat the injection with the other lobe.
   (1.2.4) Injected thymus scaffolds are transferred to the top chamber of a 12-well plate. Add 2 mL of T cell culture medium (RPMI-1640+10% FBS with other supplements, plus 1-10 ng/mL of IL-7 and 1-10 ng/mL of Flt3-L). Culture the scaffolds in 37° C. incubator with 5% CO2 for 1-14 days before transplantation (thymus engraftment can also be performed in the same day as the thymus organoid reconstruction). The in vitro incubation time allows the attachment of the TEPCs to extracellular matrix. It also facilitates monitoring to ensure that there is no contamination from bacteria or other microbes.

In some embodiments, seeding progenitor cells may involve injecting the progenitor cells into the thymus organoid. In an example, the method may comprise co-injecting into a de-cellularized mouse thymus scaffolds with iPSC-TECs and CD34+HSCs isolated from UCB. The resulting thymus organoids can be cultured in the top chambers of transwell culture system for a period of time, e.g., up to 4 weeks or more. If needed, other cells can be injected into the thymus organoid. Examples may include, for example, mesenchymal stem cells or any other cell that commonly exists in the thymus and is not directly produced from TEPCs, such as endothelial cells and dendritic cells, among others. Shown below is an example protocol for culturing of human thymus organoids in fluidic chips (flow cells) to support human T cell generation in vitro:

(1) Microfluidic chips used in these studies are designed based on the generic Aline chip design, and manufactured by ALINE, Inc. (Rancho Dominguez, Calif.). Briefly, the chips (75 mm L×25 mm W) consist of a 5-layer design and feature an 8-10 μm porous membrane. The acrylic top layer includes 4 straight barbs designed for the influx and efflux of media. The two-chambered system, separated by the porous membrane allowed for concurrent culturing of the human thymus organoids in the bottom chamber while also providing a constant flow of media across the top chamber.

(2) Thymus organoids within the microfluidics chip were run using a Multi-Syringe Programmable Syringe Pump (available from Braintree Scientific, Braintree, Mass.) loaded with T cell differentiation medium (STEMSPAN SFEM II Base Media supplemented with hIL-7—1 ng/mL, hFLT3L—100 ng/mL, HKGS—(100×, LIFE TECHNOLOGIES), hSCF—100 ng/mL, hTPO—50 ng/mL). Experiments were run at a flow rate of 80 μl/hr (flow rate of 20-200 μl/hr can be used), using media-filled syringes of the appropriate volume and diameter based on length of study. The syringe was equipped with a 23G 0.5-inch blunt needle (available from SAI Infusion Technologies, Lake Villa, Ill.). Tygon tubing (available from Warner Instruments, Hamden Conn.) was used to connect the syringe to the input port of the chip as well as connect the chip and the efflux collection flask. Additional tubing was used to close of the remaining two ports on the chip to maintain efficient flow pressure.

(3) Cells flowing out of the thymus organoids were collected in either the collagen I matrix disc housed in the same flow cell or a T-25 tissue culture flask (available from Corning Incorporated, Kennebunk, Me.) that contained 3 mL of RPMI culture media supplemented with Mouse T-Activator CD3/CD28 DYNABEADS (available from Life Technologies, Carlsbad, Calif.) and recombinant mIL-2 (available from StemCell Technologies, Cambridge, Mass.). All experiments were conducted at 37° C. and 5% CO₂.

(4) Cells were characterized by staining with fluorochrome-conjugated antibodies binding to specific surface markers and analysis with flow cytometry.

In some embodiments, preparing the cells from the thymus organoid (i.e., thymic emigrant cells) may comprise egressing or isolating the cells from the thymus organoid to obtain the thymic emigrant cells. The egressing of the cells from the thymus organoid may be observed under direct visualization using, for example, a dissecting microscope. Isolating the thymic emigrant cells from the thymus organoid may be carried out in any suitable manner. For example, the method may comprise gently removing the egressing cells by removing the media from the culture of the thymus organoid. Preferably, the isolating of the thymic emigrant cells from the thymic organoid may be carried out under direct visualization using, for example, a dissecting microscope. Preferably, the thymic emigrant cells are isolated without aspirating or disrupting the thymic organoid. The method may comprise replacing the media that was removed from the thymic organoid culture with fresh media. The thymic organoid may, subsequently, be observed for the egress of further thymic emigrant cells.

The thymic emigrant cells may be CD4−, CD8−, CD4+, CD8+, CD4−/CD8−, CD4+/CD8+, CD4+/CD8−, CD4−/CD8+, D45−, or CD45+. Alternatively or additionally, the thymic emigrant cells may be any one or more of CD45+, CD3+, CD45+/CD3+, CD62L+, CD69−, CD62L+/CD69−, CD62L−, CD69+, CD62L−/CD69+, CD45RA+CD45RO−, CD3+CD4+, and/or CD3+CD8+. In some embodiments, the thymic emigrant cells may be or include T-helper cells and subsets thereof, including the CXCR3+CCR6−Th1, CXCR3−CCR6+Th17, and CXCR3−CCR6−Th2 cells, as well as CD4+FoxP3+T-regulatory cells (Tregs), the critical population of CD4+ T cells responsible for maintaining immune tolerance. In other embodiments, the thymic emigrant cells may be or include CD4+T-helper cells, CD8+ cytotoxic TCRαβ+ T cells, or CRγδ+ T cells.

The method may further comprise differentiating the thymic emigrant cells into any desired type of cell of the T cell lineage. Examples of cell types which may be prepared by differentiating the thymic emigrant cells include, but are not limited to, natural killer T (NKT) cells, T cells (e.g., naive T cells, regulatory T-cells, T stem cell memory cells, effector T cells, effector memory RA cells (EMRA), Th1 cells, Th2 cells, or Th17 cells). The thymic emigrant cells prepared by the inventive methods may be useful for preparing cells for adoptive cell therapies.

The population of thymic emigrant cells can be a heterogeneous population comprising the thymic emigrant cells in addition to a cell other than a thymic emigrant cell, e.g., a PBMC, a B cell, a macrophage, a neutrophil, an erythrocyte, a hepatocyte, an endothelial cell, an epithelial cells, etc. Alternatively, the population of cells can be a substantially homogeneous population, in which the population comprises mainly of (e.g., consisting essentially of) thymic emigrant cells.

### Humanized Animals

Thymic organoids described herein can be used to generate humanize animals. Such animals can be used as model system to study human physiology and diseases. The animals can also be used to produce therapeutic cells for human uses.

While animal models have contributed significantly to understanding of human physiology and diseases, one of the major hurdles in translating these findings into clinic is the existence of species-specific differences between humans and animals. Humanized mice, in which human cells of hematopoietic lineage are transplanted into NOD.scid.IL2rg^{null} (NSG) or other immunodeficient IL2rg^{null} mouse lines, are powerful models that are used broadly to overcome these challenges. Hematopoietic humanized mice not only can support the long-term survival of the engrafted human cells, but also functionally recapitulate human immune responses to great extents, permitting their use as preclinical models for study of various human pathological conditions, such as infectious diseases, cancer, and autoimmunity. A comparison with other humanize mouse models of human hematopoietic cells illustrates their differences and demonstrates the benefits of the methods and models provided herein.

Based on the type of human hematopoietic cells used and the route of engraftment, current humanized mice can be generally grouped into three types. The first type generated is the hu.PBL model, in which human peripheral blood mononuclear cells (PBMCs) are intravenously (i.v.) injected in NSG mice. While engraftment of both lymphoid and myeloid cells can be readily established, hu.PBL mice develop lethal, xenogeneic graft-versus-host disease (xGVHD) within weeks due to the presence of host cell-reactive human T cells. The development of xGVHD limits the experimental time window to less than 3-4 weeks, and can complicate the interpretation of experimental findings profoundly. To overcome these defects, the hu.SRC (scid-repopulating cell) model was developed, in which recipient mice are infused with CD34+ hematopoietic stem cells. Human cells of most of the hematopoietic lineages are effectively generated in these mice, with the exception of T cells. Because of the species-related differences (e.g. growth factors and cytokines) between mouse and human, the endogenous murine thymus in hu.SRC mice cannot fully support the development of functional human T cells. To achieve robust human T cell development, the hu.BLT (or BLT for bone marrow, liver and thymus) model was developed, in which pieces of human fetal thymus and liver are co-transplanted underneath the kidney capsules of immunodeficient IL2rg^{null} recipients, together with i.v. infusion of CD34+HSCs from the same fetal donor. However, like the hu.PBL model, high incidences of xGVHD are observed in hu.BLT mice, making them unfit for long-term studies. Ethical issues regarding the use of human fetal tissue further limit their wide application in preclinical studies. Hence, the field is in dire need of novel murine models that can support the development of self-tolerant yet functional human T cells to mediate long-term adaptive immune responses.

The thymus gland does not contain HSCs capable of self-renewal, instead relying on the recruitment of common lymphocyte progenitors (CLPs) from the bone marrow to maintain long-term thymopoiesis. Once inside the thymus, CLPs undergo a series of differentiation events, such as lineage specification and positive and negative selection, to become mature T cells. Thymic epithelial cells, the predominant population of cells in the thymus stroma, play important regulatory roles throughout thymopoiesis. TECs within the cortical region (cTECs) provide key signals for T cell fate specification and positively select T cells that can functionally interact with antigen present cells (APCs). TECs within the medullary region (mTECs), on the other hand, possess the unique characteristic of expressing and presenting tissue specific self-antigens (TSAs). These mTECs are critical for eliminating T cells with high auto-reactivity in the thymus, and thus maintaining immunologic self-tolerance.

Functional thymus organoids can be constructed by repopulating decellularized thymus scaffolds with isolated murine TECs. See, e.g., Tajima, A., Pradhan, I., Geng, X., Trucco, M. & Fan, Y. Construction of Thymus Organoids from Decellularized Thymus Scaffolds. Methods in molecular biology 1576, 33-42, doi:10.1007/7651_2016_9 (2019), and Hun, M. et al. Native thymic extracellular matrix improves in vivo thymic organoid T cell output, and drives in vitro thymic epithelial cell differentiation. Biomaterials 118, 1-15, doi:10.1016/j.biomaterials.2016.11.054 (2017). The three-dimensional network of extracellular matrix in the decellularized thymus scaffolds can maintain the long-term survival and function of the TECs both in vitro and in vivo. When transplanted under the kidney capsule of athymic nude mice, tissue-engineered murine thymus organoids can support the generation of a diverse and functional T cell repertoire in the recipient mice (Fan, Y. et al. Bioengineering Thymus Organoids to Restore Thymic Function and Induce Donor-Specific Immune Tolerance to Allografts. Mol Ther 23, 1262-1277, doi:10.1038/mt.2015.77 (2015)). Thus, thymus organoids bioengineered with isolated TECs could offer complete physiologic thymic functions. However, ethical concerns and shortage of human thymus donor tissues prohibit the wide use of isolated human TECs for reconstituting the thymus function in humanized mice.

In one embodiment, this document discloses an approach to circumvent these empirical and ethical challenges through the use of induced pluripotent stem cells (iPSCs). iPSCs are stem cells reprogrammed from somatic cells by transiently overexpressing the Yamanaka factors (Oct3/4, Sox2, Klf4 and c-Myc). See Takahashi, K. & Yamanaka, S. Induction of pluripotent stem cells from mouse embryonic and adult fibroblast cultures by defined factors. Cell 126, 663-676, doi:10.1016/j.cell.2006.07.024 (2006). Similar to pluripotent embryonic stem cells (ESCs), iPSCs can be induced to differentiate into specific cell types (e.g. pancreatic islet beta cells, liver cells, neuronal cells, cardiomyocytes, etc.), and have been widely used in regenerative medicine research. TECs derived from human iPSCs can serve as a renewable source of cells for the generation of functional thymus organoids. Significant progress has been made in deriving TECs from human pluripotent stem cells (hPSCs, both embryonic and induced).

When transplanted in athymic nude mice, hPSC-derived TEPCs undergo further maturation to support the development of polyclonal mouse T cells. Similar approaches were used to differentiate human iPSCs into TEPCs. To further improve the TEPC differentiation from iPSCs, Chhatta et al. transduced differentiating iPSCs with lentiviral vectors encoding FoxN1, a master transcription regulator for TEC development (Chhatta, A. R. et al. De novo generation of a functional human thymus from induced pluripotent stem cells. J Allergy Clin Immunol 144, 1416-1419 e1417, doi:10.1016/j.jaci.2019.05.042 (2019)). Like hESC-derived TEPCs, iPSC-TEPCs can support the de novo generation of mouse T cells in nude mice. However, whether these iPSC-derived TEPCs can support the differentiation of human T cells from HSCs had not been examined.

This document describes the generation of functional human thymus organoids from human iPSCs that can support the development of human T cells from CD34+HSCs both in vitro and in vivo. For instance, as shown in the examples below, human iPSCs embedded in 3-D hydrogel capsules were subjected to an optimized multistep differentiation protocol to generate human TEPC aggregates. Human thymus organoids were constructed by repopulating decellularized murine thymus scaffolds with a combination of iPSC-TEPC aggregates and CD34+HSCs. When engrafted in hematopoietic humanized mice (designated as hu.Thor), iPSC-derived human thymus organoids support the generation of a diverse population of T cells that can mount robust alloreactive responses and effectively reject allogeneic teratomas. Sera of hu.Thor mice contain subsets of IgG, suggesting the occurrence of T cell-dependent immunoglobulin class switching in B cells. In addition, antigen (Ag)-specific IgGs against diphtheria toxoid are generated upon vaccination. Taken together, these data suggest that iPSC-derived thymus organoids can support the development of a functional human T cell compartment in hu. Thor mice.

Functional human thymus organoids disclosed herein can be tissue engineered from any suitable human pluripotent stem cells, including inducible pluripotent stem cells derived from adult cells and human embryonic stem cells. Transplantation of hPSC-derived thymus organoids in preconditioned immunodeficient mice, in conjunction with CD34+HSCs isolated from the umbilical cord blood both support the engraftment of HSCs in mice and establish humoral and cellular adaptive immunological responses. This approach provides a platform to generate customized humanized mice for recapitulating the adaptive immune system of an individual patient.

In some example, this document discloses the generation of a novel humanized mouse model that is capable of modeling a complete immune compartment in tandem with a diverse and functional T cell response. The crux of this model is the human iPSC-derived thymus organoid that can support the development of functional human T cells from CD34+HSCs. Accordingly, the humanized mouse model can be used to model conditions and discords that are characterized with severe defects in thymic function and related life-threatening immunodeficient conditions, such as primary DiGeorge syndrome and acquired immunodeficient disorders (AIDS).

### Personalized Medicine

The thymus organoid or animal model described in this document can be used for personalized medicine. More specifically, the thymus organoid or animal model can be used as an immune model for a specific patient with a particular disorder in testing how the patient may respond to certain therapy or drug for treating the disorder. In that case, cells from the patent can be used to generate a thymus organoid in the manner disclosed herein, and the thymus organoid can be further implanted in a suitable animal. Both the thymus organoid and the animal can then be used to assess the therapy or drug. In one example, when the disorder is a tumor, a sample or cells from the patient's tumor can be implanted in the animal model in vivo or co-cultured with the thymus organoid in vitro. The growth of the tumor cells in the animal or in the culture can be examined. Increased cell death, lack of growth, or described size of the implanted or cultured tumor sample as compared to a proper control indicates that the therapy or drug is suitable for treating the patient.

Humanized mouse model that has recently gained a lot of traction in cancer research is the personalized xenogenic humanized mice, in which immunocompromised mice are engrafted with PBMCs and tumor cells from the same patient. This is a powerful tool for drug testing, but as other hu.PBMC models, they will develop xenogenic graft-vs-host disease), which will limit the window of time for experiments. The humanized mouse model described in this document does not have these issues and therefore addresses the need for a more suitable model.

### Drug Evaluation and Discovery

The thymus organoid or non-human animal described above can be used in evaluating or determining whether a test compound or candidate compound (e.g., a drug candidate) can be used for treating a condition or a disorder. Alternatively, the thymus organoid or non-human animal can be used for determining a prognosis of a disorder or condition in a subject.

The evaluation methods described herein are useful to identify agents that can modulate (either promote or suppress) a disease or disorder. For example, the evaluation methods can be used to identify whether a subject has a risk of developing a disease or disorder associated with compromised immune function (e.g., inflammation, cancer, autoimmune disorder, or infection) in response to an agent. The assays can also be used to determine whether a subject is suitable to be administered with an agent (e.g., an agonist, antagonist, peptidomimetic, protein, peptide, nucleic acid, small molecule, or other drug candidate) to treat such a disorder. Information obtained from practice of the above assays is useful in prognostication, identifying progression of, and clinical management of diseases and other deleterious conditions affecting an individual's health status. In preferred embodiments, the diagnostic assays provide information useful in prognostication, identifying progression of and management of conditions that are characterized by inflammation, cancer, autoimmune disorder, or infection. The information more specifically assists a clinician in designing treatment regimes to treat or prevent such conditions.

A test compound can be evaluated by incubating the thymus organoid in a test medium containing the test compound for a period of time; and determining an effect on cells in the thymus organoid or cells emigrate from the thymus organoid. A test compound can also be administered to the non-human animal. After a period of time, the animal or its cells can be examined for effects by the test compound. To that end, one can examine effects of the compound or composition on the thymus organoid in the animal, on cells in the thymus organoid, or on cells emigrate from the thymus organoid.

In one embodiment, the method may include examining activation of T cells in or from the thymus organoid. T cell activation can be determined during and/or following co-culturing of the compound and the thymus organoid. Suitable assays for T cell activation include DNA replication assays (e.g., ³H-thymidine incorporation), extracellular and/or cytokine production assays (e.g., ELISA, flow cytometry, and the like), and T cell activation marker assays (e.g., flow cytometry).

In some example, T cell activation can be measured by extracellular or intracellular cytokine production, such as, IFNγ and/or IL-2 production, and the like. Extracellular cytokine production can be measured by determining changes in levels of one or more cytokines in culture media. Typically an immunoassay (e.g., ELISA assay, sandwich assay, immunoprecipitation assay, or Western blotting) can be used, although other assays can also be suitable. (See, e.g., Harlow and Lane, Using Antibodies, A Laboratory Manual, Cold Spring Harbor Laboratory, New York (1999)) For intracellular cytokine levels, immunoassays or other assays can be used. The T cells can optionally be separated from the organoid or animal (e.g., by collection based on expression of T cell markers), prior to assay for intracellular cytokine levels. (See, e.g., Harlow and Lane, supra). In additional embodiments, T cell activation can be determined by modulation of T cell activation markers. Such markers include, for example, CD25, CD69, CD44, CD125, and the like. The modulation of T cell activation markers can be measured, for example, by determining changes in protein levels or mRNA levels. Changes in protein levels can be determined by flow cytometry using labeled antibodies against the T cell activation markers, transcription factors or other proteins associated with T cell activation, by immunoassay, such as, ELISA or Western blotting, and the like. Changes in mRNA levels can be determined for the message encoding the T cell activation markers, transcription factors, and the like. mRNA levels can be determined by, for example, Northern blotting, polymerase chain reaction (e.g., RT-PCR), other hybridization assays (e.g., assays using probe arrays, and the like), or other assays. (See, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

In one example, a tumor sample/tissue can be co-cultured with the thymus organoid or transplanted in the non-human animal. A test compound or test composition may be identified as a candidate compound or a candidate composition suitable for treating the tumor if, after administering the compound/composition, a level of tumor cell death or a level of immune cell infiltration (e.g., T cell infiltration) in the tumor sample or tissue is higher than a control level. Alternatively, the test compound or test composition may be identified as suitable for treating the tumor if, after administering the compound/composition, the tumor growth level is lower than a control level.

A candidate compound/composition identified by the evaluation method can be further tested to confirm its therapeutic effect or modified to optimize its effect and limit any side effects, and then formulated as a therapeutic agent. Therapeutic agents thus identified can be used in a therapeutic protocol to treat the diseases

Examples of such a test compound include small organic or inorganic molecules, proteins, peptides, peptidomimetics, polysaccharides, nucleic acids, nucleic acid analogues and derivatives, or peptoids. Candidate compounds to be screened (e.g., proteins, peptides, peptidomimetics, peptoids, antibodies, small molecules, or other drugs) can be isolated from naturally occurring substances or obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann et al. 1994, J. Med. Chem. 37:2678-2685; and Lam, 1997, Anticancer Drug Des. 12:145. Examples of methods for the synthesis of molecular libraries can be found in, e.g., DeWitt et al., 1993, PNAS USA 90:6909; Erb et al., 1994, PNAS USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994 J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (U.S. Pat. No. 5,223,409), spores (U.S. Pat. No. 5,223,409), plasmids (Cull et al., 1992, PNAS USA 89:1865-1869), or phages (Scott and Smith 1990, Science 249:386-390; Devlin, 1990, Science 249:404-406; Cwirla et al., 1990, PNAS USA 87:6378-6382; Felici 1991, J. Mol. Biol. 222:301-310; and U.S. Pat. No. 5,223,409).

### Rejuvenating Thymus Function

Age-associated thymus involution causes decreased T cell output, leading to constriction of T cell diversity and compromised adaptive immune responses. The thymus gland is also extremely sensitive to external insults, such as chemotherapy, irradiation and infections, which can cause irreversible damages. Thus, there is a strong clinical need to rejuvenate thymus function for patients in need.

Rejuvenating thymus function have broad clinical impacts for treatment of thymus deficiency. The thymus organoid described herein can be used to rejuvenate thymus function in a subject in need thereof. To that end, thymus organoids can be bioengineered from the subject's own PSCs (autologous) or PSCs from a matched donor (allogeneic). This document shows here that functional human thymus organoids are capable of supporting the development of human T cells from human HSCs both in vitro and in vivo can be tissue-engineered from human iPSCs.

In some embodiments, the document provides a method for rejuvenating thymus function in a subject in need thereof. The method includes transplanting thymus organoids described herein into the subject. The thymus organoids can be allogeneic or preferably autologous. In some embodiments immune suppressive treatments can also administered as needed.

### Cellular Immunotherapy.

The present document also relates to agents, methods and compositions to confer and/or increase immune responses mediated by cellular immunotherapy, such as by adoptively transferring antigen-specific genetically modified subsets of lymphocytes. Such an adoptive cell transfer or adoptive cell therapy (ACT) represents a promising therapeutic approach for the treatment of cancer patients. The document provides compositions comprising genetically modified lymphocytes that express chimeric antigen receptors having the ability to modulate the immune system and the innate and adaptive immune response. The disclosed agents, methods, and compositions provide genetically engineered lymphocytes with enhanced anti-tumor functions as well as methods of developing such lymphocytes.

Genetically modified immune function cells, such as T cells and NK cells engineered to express foreign antigen receptors are effective immunotherapeutic for cancer and infectious diseases. Isolation of autologous antigen specific immune cells, such as T cells, for therapeutic application is a laborious task, and is not possible where such cells are absent or rare. Therefore, strategies have been developed to genetically transfer immune receptors specific to tumor or virus into patients' T cells. To this end, antigen receptors have been constructed that join antigen (Ag)-recognition domains to signaling domains of the TCR or Fc receptor. T cells expressing such antigen receptors can recapitulate the immune specific responses mediated by the introduced receptor.

Chimeric antigen receptors (also known as chimeric immunoreceptors, chimeric T cell receptors or artificial T cell receptors) are receptor proteins that have been engineered to give T cells the new ability to target a specific protein. The receptors are chimeric because they combine antigen binding and T-cell activating functions into a single receptor. In addition to antigen-binding sites, a CAR can have one or more function domains.

The thymus organoid and humanized animal described herein can be used to make genetically modified immune function cells, such as T cells and NK cells, expressing a CAR. In some embodiments, a lenti- or retro-viral vector containing one or more transgenes encoding, e.g., a specific T-cell receptor (both the alpha and beta chains) can be used to transduce CD34+ bone marrow progenitor cells. The transduced cells can be then seeded in the thymus organoid described herein and further differentiated to T cells and expanded using the thymus organoid system in vitro (e.g., the fluidic chip), or in vivo (in the humanized mouse). Using this approach, a sufficient amount of CAR-T cells can be made for treating disorders such as infection, cancer or a tumor.

Transgenes can be introduced into target cells using various methods. These methods include, but are not limited to, transduction of cells using integration-competent gamma-retroviruses or lentivirus, and DNA transposition.

A wide variety of vectors can be used for the expression of the transgenes. The ability of certain viruses to infect cells or enter cells via receptor-mediated endocytosis, and to integrate into a host cell genome and express viral genes stably and efficiently have made them attractive candidates for the transfer of foreign nucleic acids into cells. Accordingly, in certain embodiments, a viral vector is used to introduce a nucleotide sequence encoding one or more transgenes or fragment thereof into a host cell for expression. The viral vector may comprise a nucleotide sequence encoding one or more transgenes or fragment thereof operably linked to one or more control sequences, for example, a promoter. Alternatively, the viral vector may not contain a control sequence and will instead rely on a control sequence within the host cell to drive expression of the transgenes or fragment thereof. Non-limiting examples of viral vectors that may be used to deliver a nucleic acid include adenoviral vectors, adeno-associated virus (AAV) vectors, and retroviral vectors.

Physical methods for introducing a polynucleotide into a host cell include calcium phosphate precipitation, lipofection, particle bombardment, microinjection, electroporation, and the like. Methods for producing cells comprising exogenous vectors and/or nucleic acids are well known in the art. See, for example, Sambrook et al. (2001, Molecular Cloning: A Laboratory Manual, Cold Spring Harbor Laboratory, New York).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as an in vitro and in vivo release vehicle is a liposome (e.g., an artificial membrane vesicle).

In the case where a non-viral delivery system is used, an exemplary delivery vehicle is a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, bound to a liposome via a binding molecule that is associated with both the liposome and the oligonucleotide, entrapped in a liposome, in a complex with a liposome, dispersed in a solution containing a lipid, mixed with a lipid, combined with a lipid, contained as a suspension in a lipid, content or in a complex with a micelle, or associated otherwise with a lipid. The compositions associated with lipids, lipids/DNA or lipids/expression vector are not limited to any particular structure in solution. For example, they can be present in a bilayer structure, as micelles, or with a “collapsed” structure. They can also be simply interspersed in a solution, possibly forming aggregates that are not uniform in size or shape. Lipids are fatty substances that can be natural or synthetic lipids. For example, lipids include fatty droplets that occur naturally in the cytoplasm as well as the class of compounds containing long-chain aliphatic hydrocarbons and their derivatives, such as fatty acids, alcohols, amines, amino alcohols, and aldehydes.

Regardless of the method used to introduce exogenous nucleic acids into a host cell, the presence of the recombinant DNA sequence in the host cell can be confirmed by a series of tests. Such assays include, for example, molecular biology assays well known to those skilled in the art, such as Southern and Northern blot, RT-PCR and PCR; biochemical assays, such as the detection of the presence or absence of a particular peptide, for example, by immunological means (ELISA and Western blot) or by assays described herein to identify agents that are within the scope of this document.

### Pharmaceutical Composition

The therapeutic cells (e.g., thymic emigrant cells, CAT-T cells, or NK-T cells) described above can be formulated into a composition, such as a pharmaceutical composition. Such a pharmaceutical composition may comprise any of the populations of cells described herein and a pharmaceutically acceptable carrier. In some embodiments, the pharmaceutical composition can comprise a population of cells and another pharmaceutically active agent(s) or drug(s), such as a chemotherapeutic agents. Preferably, the carrier is a pharmaceutically acceptable carrier suitable for the particular population of cells under consideration. Such pharmaceutically acceptable carriers are well-known to those skilled in the art and are readily available to the public. Suitable formulations may include any of those for parenteral, subcutaneous, intratumoral, intravenous, intramuscular, intraarterial, intrathecal, or interperitoneal administration.

In one example, the composition of cells can be administered by injection, e.g., intravenously. When the population of therapeutic cells is to be administered, the pharmaceutically acceptable carrier for the therapeutic cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution, PLASMA-LYTE A, about 5% dextrose in water, or Ringer's lactate. In an embodiment, the pharmaceutically acceptable carrier is supplemented with human serum albumin.

The amount or dose of the population of therapeutic cells or pharmaceutical composition administered (e.g., numbers of cells when the population of cells is administered) should be sufficient to effect, e.g., a therapeutic or prophylactic response, in the patient over a reasonable time frame. For example, the dose of the population of cells or pharmaceutical composition should be sufficient to treat or prevent a condition in a period of from about 2 hours or longer, e.g., 12 to 24 or more hours, from the time of administration. In certain embodiments, the time period could be even longer. The dose can be determined by the efficacy of the particular population of cells or pharmaceutical composition administered and the condition of the patient, as well as the body weight of the patient to be treated. Assays for determining an administered dose are known in the art. For example, an assay, which comprises comparing the extent to which target cells are lysed upon administration of a given dose of such therapeutic cells to a mammal among a set of mammals of which is each given a different dose of the cells, could be used to determine a starting dose to be administered to a patient. The extent to which target cells are lysed upon administration of a certain dose can be assayed by methods known in the art.

The dose of the population of cells or pharmaceutical composition also will be determined by the existence, nature and extent of any adverse side effects that might accompany the administration of a particular population of cells or pharmaceutical composition. Typically, the attending physician will decide the dosage of the population of cells or pharmaceutical composition with which to treat each individual patient, taking into consideration a variety of factors, such as age, body weight, general health, diet, sex, population of cells or pharmaceutical composition to be administered, route of administration, and the severity of the condition being treated.

This document also provides a method of treating or preventing a condition in a mammal. The method comprises administering the therapeutic cells described above to the mammal in an amount effective to treat or prevent the condition in the mammal. In an embodiment of this document, the condition is a cancer, an immunodeficiency, an autoimmune condition, an infection, or a blood condition.

Examples of the cancer may include, but not limited to, any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

Examples of the immunodeficiency may be any condition in which the body's ability to defend itself against outside pathogens is disrupted. The immunodeficiency may be any condition in which a patient's immune system is compromised and in need of reconstitution after immunodeployment due to irradiation or chemotherapy. Immunodeficiency may include, for example, a depleted adaptive immune system in the elderly population. To that end, the thymic organoid described in this document may produce therapeutic cells which may be useful for the treatment of both primary and secondary immuno-deficiencies. Examples of immuno-deficiencies which may be treated or prevented include, but are not limited to X-linked agammaglobulinemia (XLA), variable immunodeficiency (CVID), severe combined immunodeficiency (SCID), AIDS, and hepatitis.

The autoimmune condition may be any condition in which the body's immune system attacks healthy cells. The thymic organoid described in this document may produce therapeutic cells which may be useful for the treatment of autoimmune conditions. Examples of autoimmune conditions which may be treated or prevented include, but are not limited to, rheumatoid arthritis, lupus, type 1 diabetes, multiple sclerosis, celiac disease, temporal arteritis, vasculitis, alopecia areata, ankylosing spondylitis, Sjogren's syndrome, and polymyalgia rheumatic.

The infection may be an infectious condition, for example, a viral infection, a bacterial infection, a fungal infection, or a protozoan infection. As used herein, “viral infection” means a condition that can be transmitted from person to person or from organism to organism, and is caused by a virus. In an embodiment of this document, the viral condition may be caused by a virus selected from the group consisting of herpes viruses, pox viruses, hepadnaviruses, papilloma viruses, adenoviruses, coronoviruses, orthomyxoviruses, paramyxoviruses, flaviviruses, and caliciviruses. For example, the viral condition may be caused by a virus selected from the group consisting of respiratory syncytial virus (RSV), influenza virus, herpes simplex virus, Epstein-Barr virus, varicella virus, cytomegalovirus, hepatitis A virus, hepatitis B virus, hepatitis C virus, human immunodeficiency virus (HIV), human T-lymphotropic virus, calicivirus, adenovirus, and Arena virus. The viral infection may be, for example, influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, HIV/AIDS, and mononucleosis.

The blood condition may be any non-cancerous condition that affects the blood. The blood condition may be, for example, cytopenia (e.g., anemia, leukopenia, and neutropenia), bleeding disorders such as hemophilia, and blood clots.

### Definitions

The term “antigen receptor” or “antigen recognizing receptor” as used herein refers to a receptor that is capable of activating an immune cell (e.g., a T-cell) in response to antigen binding. In particular, the term “antigen receptor” includes engineered receptors, which confer an arbitrary specificity onto an immune effector cell such as a T cell. An antigen receptor according to this document may be present on T cells, e.g. instead of or in addition to the T cell's own T cell receptor. Such T cells do not necessarily require processing and presentation of an antigen for recognition of the target cell but rather may recognize preferably with specificity any antigen present on a target cell. Preferably, said antigen receptor is expressed on the surface of the cells. Specifically, the term includes artificial or recombinant receptors comprising a single molecule or a complex of molecules which recognize, i.e. bind to, a target structure (e.g. an antigen) on a target cell (e.g. by binding of an antigen binding site or antigen binding domain to an antigen expressed on the surface of the target cell) and may confer specificity onto an immune effector cell such as a T cell expressing said antigen receptor on the cell surface. Preferably, recognition of the target structure by an antigen receptor results in activation of an immune effector cell expressing said antigen receptor. An antigen receptor may comprise one or more protein units said protein units comprising one or more domains as described herein. The term “antigen receptor” preferably does not include naturally occurring T cell receptors. According to this document, the term “antigen receptor” is preferably synonymous with the terms “chimeric antigen receptor”, “chimeric T cell receptor” and “artificial T cell receptor.” Exemplary antigen recognizing receptors may be native or genetically engineered TCRs, or genetically engineered TCR-like mAbs (Hoydahl et al. Antibodies 2019 8:32) or CARs in which a tumor antigen-binding domain is fused to an intracellular signaling domain capable of activating an immune cell (e.g., a T-cell).

The term “Chimeric Antigen Receptor” or “CAR” refers to a set of polypeptides, typically two in the simplest embodiments, which when in an immune effector cell, provides the cell with specificity for a target cell and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain, e.g., comprise a chimeric fusion protein. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen-binding domain to an intracellular signaling domain. In one aspect, the stimulatory molecule of the CAR is the zeta chain associated with the T cell receptor complex (e.g., CD3 zeta). In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137), CD27, and/or CD28.

The term “immune cells” refers to cells of hematopoietic origin that are involved in the specific recognition of antigens. Immune cells include antigen presenting cells (APCs), such as dendritic cells or macrophages, B cells, T-cells, NK cells such as NK-92 cells, etc. T-cells include Teff cells and Treg cells.

The term “lymphocyte” as used herein can include natural killer (NK) cells, T cells, or B cells. NK cells are a type of cytotoxic (cell toxic) lymphocyte that represent a major component of the inherent immune system. NK cells reject tumors and cells infected by viruses through the process of apoptosis or programmed cell death. T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. There are several types of T-cells, namely: Helper T-cells (e.g., CD4+ cells, effector T_{EFF} cells), Cytotoxic T-cells (also known as TC, cytotoxic T lymphocyte, CTL, T-killer cell, cytolytic T cell, CD8+ T-cells or killer T cell), Memory T-cells ((i) stem memory T_{sccm} cells, like naive cells, are CD45RO−, CCR7+, CD45RA+, CD62L+(L-selectin), CD27+, CD28+ and IL-7Ra+, but they also express large amounts of CD95, IL-2R13, CXCR3, and LFA-1, and show numerous functional attributes distinctive of memory cells); (ii) central memory T_{sccm} cells express L-selectin and are CCR7+ and CD45RO+ and they secrete IL-2, but not IFNγ or IL-4, and (iii) effector memory TEM cells, however, do not express L-selectin or CCR7 but do express CD45RO and produce effector cytokines like IFNγ and IL-4), Regulatory T-cells (Tregs, suppressor T cells, or CD4+CD25+ regulatory T cells), Natural Killer T-cells (NKT), and Gamma Delta T-cells. T cells found within tumors are referred to as “tumor infiltrating lymphocytes” or “TIL.” B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

The term “stem cell” refers to a cell capable of self-replication and pluripotency or multipotency. Typically, stem cells can regenerate an injured tissue. Stem cells herein may be, but are not limited to, embryonic stem (ES) cells, induced pluripotent stem cells or tissue stem cells (also called tissue-specific stem cell, or somatic stem cell).

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

“Peripheral blood cells” refer to the cellular components of blood, including red blood cells, white blood cells, and platelets, which are found within the circulating pool of blood.

“Hematopoietic stem and progenitor cells” or “hematopoietic precursor cells” refers to cells that are committed to a hematopoietic lineage but are capable of further hematopoietic differentiation and include hematopoietic stem cells, multipotential hematopoietic stem cells (hematoblasts), myeloid progenitors, megakaryocyte progenitors, erythrocyte progenitors, and lymphoid progenitors. “Hematopoietic stem cells (HSCs)” are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells).

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, mouse, a non-human primate (for example, a monkey, such as a cynomolgus monkey, chimpanzee, etc) and a human). The subject may be a human or a non-human. In more exemplary aspects, the mammal is a human. In some embodiments, the subject is a human. In some embodiments, the subject has a cancer. In some embodiments, the subject is immune-depleted.

“Treating” or “treatment” as used herein refers to administration of a compound or agent to a subject who has a disorder with the purpose to cure, alleviate, relieve, remedy, delay the onset of, prevent, or ameliorate the disorder, the symptom of a disorder, the disease state secondary to the disorder, or the predisposition toward the disorder.

The terms “treat” and “prevent” do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a condition in a patient. Furthermore, the treatment or prevention provided by the inventive method can include treatment or prevention of one or more conditions or symptoms of the condition being treated or prevented. For example, treatment or prevention can include promoting the regression of a tumor. Also, for purposes herein, “prevention” can encompass preventing the recurrence of the condition, delaying the onset of the condition, or a symptom or condition thereof.

An “effective amount” or “therapeutically effective amount” refers to an amount of the compound or agent (e.g., T cells or DC cells) that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route. The ability of the T cells or DC cells to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained. The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a therapeutic agent or cell within or to the subject such that it may perform its intended function

The term “autologous” refers to any material derived from the same subject or individual to which it is later to be re-introduced. For example, the autologous cell therapy method described herein involves collection of lymphocytes, immune cells, or progenitors thereof from a donor, e.g., a patient, which are then engineered to express, e.g., a CAR construct, and then administered back to the same donor, e.g., patient.

The term “heterologous” refers to any material (e.g., cells or tissue scaffold) derived from a different subject or individual. As used herein, “heterologous” or “non-endogenous” or “exogenous” also refers to any material (e.g., gene, protein, compound, molecule, cell, or tissue or tissue component) or activity that is not native to a host cell or a host subject, or is any gene, protein, compound, molecule, cell, tissue or tissue component, or activity native to a host or host cell but has been altered or mutated such that the structure, activity or both is different as between the native and mutated versions.

The term “allogeneic” refers to any material (e.g., cells or tissue scaffold) derived from one individual which is then introduced to another individual of the same species, e.g., allogeneic cell transplantation. For example, cells may be obtained from a first subject, modified ex vivo according to the methods described herein and then administered to a second subject in order to treat a disease. In such embodiments, the cells administered to the subject are allogeneic and heterologous cells.

A “vector” is a nucleic acid molecule or a particle that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment. In certain embodiments, the vector is a viral vector. Examples of viral vectors include, but are not limited to, adenovirus vectors, adeno-associated virus vectors, retrovirus vectors, gammaretrovirus vectors, and lentivirus vectors. “Retroviruses” are viruses having an RNA genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HIV type 1 and HIV type 2, equine infectious anemia virus, feline immunodeficiency virus (Hy), bovine immune deficiency virus (BIV), and simian immunodeficiency virus (SIV). In other embodiments, the vector is a non-viral vector. Examples of non-viral vectors include lipid-based DNA vectors, modified mRNA (modRNA), self-amplifying mRNA, closed-ended linear duplex (CELiD) DNA, and transposon-mediated gene transfer (PiggyBac, Sleeping Beauty). Where a non-viral delivery system is used, the delivery vehicle can be a liposome. Lipid formulations can be used to introduce nucleic acids into a host cell in vitro, ex vivo, or in vivo. The nucleic acid may be encapsulated in the interior of a liposome, interspersed within the lipid bilayer of a liposome, attached to a liposome via a linking molecule that is associated with both the liposome and the nucleic acid, contained or complexed with a micelle, or otherwise associated with a lipid.

### EXAMPLES

### Example 1 Materials and Methods

This example describes material and methods used in Examples 2-8 bellow.

Mice

All animal procedures were approved by the Allegheny General Hospital Institutional Animal Care and Use Committee. NOD.Cg.Prkdc^{scid}Il2rg^{tm1Wjl}SzJ (NSG) mice (Strain #005557) were purchased from Jackson Laboratory (Bar Harbor, Me.). Mice were 8-12 weeks old on the date of transplantation. NSG mice were housed in a pathogen-free facility. 4-8 week old FVB mice were purchased from Jackson Laboratory and were housed and cared for according to National Institutes of Health and the Association for Assessment and Accreditation of Laboratory Animal Care International.

Generation of iPS-Derived Thymic Epithelial Cells (TECs)

The stage-wise induction protocol for thymic epithelial progenitor (TEP) differentiation of hESCs was adopted from a previous study (Parent, A. V. et al. Cell stem cell 13, 219-229, doi:10.1016/j.stem.2013.04.004 (2013)). Stage 1 for definitive endoderm (DE) was carried out with Melton beta cell differentiation protocol. Stage 2 for anterior foregut endoderm (AFE) was carried out in RPMI media supplemented with 0.5% B27 (GIBCO, Gaithersburg, Md.). For stage 2 (AFE), the following factors were used: 100 ng/ml ActivinA on day 5, 0.25 μM Retinoic Acid on days 5-7, 50 ng/ml BMP4 (MILTENYI BIOTEC, Auburn, Calif.) on days 6-7, and 5 μM LY364947 (MILLIPORE SIGMA, Burlington, Mass.) on days 6-7. Stages 3 and 4 for ventral pharyngeal endoderm (VPE) and TEP were carried out in DMEM/F12 media (GIBCO) supplemented with 0.5% B27. For stages 3 (VPE) and 4 (TEP), the following factors were used: 50 ng/ml Wnt3A on days 8-11, 0.1 μM Retinoic Acid on days 8-11, 50 ng/ml BMP4 on days 8-11, 5 μM LY364947 on days 8-9, 50 ng/ml FGF8b (MILTENYI BIOTEC) on days 8-11, and 0.5 μM KAAD-cyclopamine (MILLIPORE) on days 8-11.

Humanized Mouse Conditioning and Cellular Preparation

Hu.SRC mice were generated via transplanting conditioned NSG mice with human umbilical cord-derived CD34+ cells. In brief, certain recipient NSG mice were given 250 μL of 2 mg/mL anti-mouse CD117 (c-kit) antibody intraperitoneally (BIOLEGEND, San Jose, Calif.) one week before transplant. At 48 and 24 hours before infusion of human cord blood cells, all NSG mice involved in the study were given 30 μg/g of busulfan intraperitoneally (SAGENT PHARMACEUTICALS, Schaumburg, Ill.).

Human umbilical cord blood was purchased from VITALANT (Pittsburgh, Pa.). Ficoll separation was utilized to separate the lymphocyte population from the blood and the CD34+ cell population was isolated via the human CD34 Microbead Kit ULTRAPURE (MILTENYI BIOTEC) and confirmed using flow cytometry staining for human CD45-APC (BD BIOSCIENCES) and human CD34-VIOBLUE (MILTENYI BIOTEC). Cells were then either frozen or cultured until transplant. On the day of transplant, 1×10⁵-1×10⁶ CD34+ cells were injected retroorbitally into recipient mice.

Human Thymic Organoid Preparation and Transplant

Murine thymus decellularization was performed using chemical detergent washing as previously described (Tajima, A., Pradhan, I., Geng, X., Trucco, M. & Fan, Y. Construction of Thymus Organoids from Decellularized Thymus Scaffolds. Methods in molecular biology 1576, 33-42, 2019). In brief, thymic glands from 3-4 week old C57BL/6J.CD45.1 mice were harvested in 0.1% sodium dodecyl sulfate (INVITROGEN, Grand Island, N.Y.) in deionized water under continuous rotation (LAB LINE, THERMO SCIENTIFIC, Waltham, Mass.) until the tissue was translucent and white in color (˜24 hours). The organs were then washed three times in phosphate-buffered saline (PBS) and subsequently incubated in 1% TRITON X-100 (SIGMA-ALDRICH, St. Louis, Mo.). After three more PBS washes, the organs were washed a final time in PBS plus penicillin/streptomycin (100 U/ml) and rotated for an additional 48 hours. The decellularized thymus scaffolds were stored in PBS at 4° C. for up to 1 month and were switched to Roswell Park Memorial Institute (RPMI)-10 culture medium supplemented with 10% fetal bovine serum, 100 U/ml Penicillin, 100 μg/ml Streptomycin, 2 mmol/11-glutamine, and 10 mmol/1 HEPES 24 hours before use.

Decellularized scaffolds were then reconstituted with isolated CD34+ umbilical cord blood cells and decapsulated thymic epithelial cells. To decapsulate TECs, alginate capsules were gently pelleted and incubated in 100 mM EDTA solution for 5 minutes at room temperature. After washing with PBE, cells were then pelleted and counted. After combining with the CD34+ cells at ˜1:10 ratio (TEC:CD34+), the mixture was then pelleted and resuspended in 40 μL of STEMSPAN SFEM II base media supplemented (STEMCELL Technologies, Vancouver, Canada) with human SCF (100 ng/mL), human FLT3L (100 ng/mL), human TPO (50 ng/mL) (MILTENYI), and human keratinocyte growth factor supplement (THERMOFISHER SCIENTIFIC, Waltham, Mass.). The cells were then injected into both lobes of the thymic scaffold with a pulled glass needle and cultured in a transwell system for 5-7 days prior to transplant. On the day of transplant, scaffolds were gently rinsed three times in saline prior to surgery. Recipient hu. Thor mice were anesthetized with isofluorane and the scaffold was transplanted beneath the left kidney capsule.

Flow Cytometric Analyses

Decapsulated thymic epithelial cells were stained with the following antibodies to determine their surface protein expression: anti-human CD45-APC, MHC Class II-PerCP-Cy5.5 (BD BIOSCIENCES), EpCAM-PE (INVITROGEN). All samples were compared to fluorochrome-matched IgG controls.

Blood was harvested from hu. Thor mice at various time points to determine both the humanization and T cell generation. Blood was stained with the following monoclonal antibodies: anti-human CD45−APC, CD3−PE, CD4−V450, CD8−FITC, and anti-mouse CD45−APC/Cy7 (BD BIOSCIENCES, San Jose, Calif.) and red blood cells were lysed. The remaining cell population was fixed in 2% paraformaldehyde and analyzed on a FACS Influx system (BD BIOSCIENCES). On days of animal sacrifice and tissue harvest, murine bone marrow and splenocytes were stained with the following additional antibodies: anti-human CD20−V450, CD14−PE, CD33−V450, CD45RA−PE, CD45RO−FITC, CD25−V450, CD11c−FITC, CD56−FITC, CD117−PE, TCRαβ−PE, TCRγδ−FITC, CCR6−BV421, and CXCR3−BV510 (BD BIOSCIENCES). Anti-human FOXP3−PE was also stained for intracellularly with the FOXP3/Transcription Factor Staining Buffer set according to manufacturer's protocol (THERMOFISHER). Additional information on the stains used can be found in Table 1. Dead cells were excluded from analyses through the use of the LIVE/DEAD Violet Fixable dead cell stain kit (THERMOFISHER). All corresponding flow cytometry analyses were performed on FLOWJO 10 (Version 10.5.3) software (Ashland, Oreg.).

TABLE 1
FACS antibodies used in hu.Thor studies

| Antibody | Species | Fluorochrome | Clone | BD Catalog # |
|---|---|---|---|---|
| CD45 | Mouse | APC-Cy7 | 30-F11 | 557659 |
| CD45 | Human | APC | HI30 | 555485 |
| CD3 | Human | PE | HIT3a | 555340 |
| CD4 | Human | V450 | RPA-T4 | 560345 |
| CD8 | Human | FITC | HIT8a | 555634 |
| CD20 | Human | V450 | L27 | 561164 |
| CD11c | Human | FITC | B-ly6 | 561355 |
| CD14 | Human | PE | MφP9 | 562691 |
| CD56 | Human | FITC | B159 | 562794 |
| CD33 | Human | V450 | WM53 | 561157 |
| CD45RO | Human | FITC | UCHL1 | 555492 |
| CD45RA | Human | PE | HI100 | 555489 |
| FoxP3 | Human | PE | PCH101 | 12-4776-42 |
| | | | | (eBioscience) |
| CD25 | Human | V450 | M-A251 | 560355 |
| CCR6 | Human | BV421 | 11A9 | 562515 |
| CXCR3 | Human | BV510 | 1C6/CXCR3 | 740183 |
| MHC Class II | Human | PerCP-Cy5.5 | G46-6 | 552764 |
| EpCAM | Human | PE | 1B7 | 12-9326-42 |
| | | | | (eBioscience) |
| TCRβ | Human | PE | T10B9.1A-31 | 561674 |
| TCRγδ | Human | FITC | B1 | 559878 |

RNA Isolation and Gene Expression Analysis

Total RNA was extracted with TRIZOL (INVITROGEN, Waltham, Mass.) and cDNA was synthesized using the SUPERSCRIPT III First-Strand synthesis system for RT-PCR (INVITROGEN). Quantitative real-time PCR was performed using the All-in-One qPCR Mix (GENECOPOEIA, Rockville, Md.) with a ROCHE LIGHTCYCLER 480 system (ROCHE APPLIED SCIENCE, Indianapolis, Ind.). Quantitative real-time PCR primer sequences used in this study are shown in Table 2. PCR reactions were performed in triplicate in at least three separate experiments. Relative gene expression was normalized to GAPDH.

TABLE 2
Human quantitative PCR primer sequences

| | | SEQ | | SEQ |
|---|---|---|---|---|
| | | ID | | ID |
| Primer | Forward Sequence | NO. | Reverse Sequence | NO. |
| Cytokeratin 5 | GAATGCAGACTCAGTGGAGAAG | 1 | CACTGCCATATCCAGAGGAAAC | 2 |
| Cytokeratin 8 | CAGGAGCTGATGAACGTCAA | 3 | CATGTTCTGCATCCCAGACT | 4 |
| Cytokeratin 17 | GATGCCGAGGATTGGTTCTT | 5 | GATCTCACTCTTGCCACTCTG | 6 |
| Cytokeratin 18 | GACCTGGACTCCATGAGAAATC | 7 | GTTGAGCTGCTCCATCTGTA | 8 |
| OCT4 | CCGAAAGAGAAAGCGAACGAG | 9 | ATGTGGCTGATCTGCTGCAGT | 10 |
| SOX2 | CCATGACCAGCTCGCAGAC | 11 | GGACTTGACCACCGAACCC | 12 |
| SOX17 | GTGGACCGCACGGAATTTG | 13 | GGAGATTCACACCGGAGTCA | 14 |
| HOXA3 | GAAAACCAGCAGCTCCAG | 15 | GCGCTCGTGTAGGCCGTG | 16 |
| EYA1 | AATGTTGGAGGTCTGCTTGG | 17 | CGGTCAGGGCTTCAATTTCG | 18 |
| FOXN1 | GACGGAGCACTTTCCTTACTT | 19 | CCTGATTTGTTCTCCACCTTCT | 20 |
| PAX9 | GTACGGTCAGGCACCAAAT | 21 | CAGCACTGTAGGTCATGTAAGG | 22 |
| PRSS16 | GGCTCAGACGAACTCCTACTA | 23 | GAGGATCCTAAAGCCTGTGTTAC | 24 |
| ACKR4 | TGCCTCAACCCAATCCTTTAT | 25 | CCACACTTTGTCTCTGTCTTCT | 26 |
| CD205 | ATGGACAGAAGTGGTGGATAAG | 27 | CCTCTCCAAATGTCACAGGAA | 28 |
| β5t | GTCTCAGCTTCAGCGTCTTT | 29 | CACCATCCAAAGGGTGTCTATC | 30 |
| AIRE | CCACCTCTTGTCAGTGCTCGG | 31 | GGGTTTAATTTCCAGGCACATG | 32 |
| CSN2 | CAGCAAGGAGAGGATGAACAC | 33 | GGGATAGGTTCAACGAATGGATAG | 34 |
| MHC Class II | AAGCAATGCAGCAGAACGC | 35 | GTAAAGCCATTAAAGCAGAAG | 36 |
| CD74 | GAACACCATGGAGACCATAGAC | 37 | AGGAGTGCCTGCTCATTTC | 38 |
| GAPDH | TGCACCACCAACTGCTTAGC | 39 | GGCATGGACTGTGGTCATGAG | 40 |

For gene expression profiling analysis, RNA was extracted from the splenocytes of hu.Thor and hu.SRC humanized mice using the TRIzol™ method (INVITROGEN), following the manufacturer's protocol. The concentration, quality, and integrity of each RNA sample was examined first by Nanodrop Spectrophotometer, followed by BIOLANALYZER 2100 (RNA Pico kit, AGILENT). Only samples with DV200 values above than 70% were used in the study. 150-500 ng of total RNA was loaded to CAR-T cell gene profiling hybridization cartridge that can capture reporter probes for 771 genes (with unique barcode) specific to various pathways of T cell function, and was characterized with nCounter Max Gen 2 System (NANOSTRING TECHNOLOGIES, Seattle, Wash.). Splenocytes isolated from NSG mice were used as negative control, in which less than 15% of the genes were above background and were excluded from the analysis. All reads were analyzed with NSOLVER 4.0 Advanced analysis software. Specifically, pathway score analysis was performed, which condense each sample's gene expression profile into a small set of pathway scores. Pathway scores are fit using the first principal component of each gene set's data. They are oriented such that increasing score corresponds to mostly increasing expression (specifically, each pathway score has positive weights for at least half its genes). Summary plots explore the joint behavior of pathways, and Covariates plots compare pathway scores to covariates.

To characterize the TCR Vβ gene family usage, total reads of Vβ genes from the T cell gene panel were calculated and percentage of individual Vβ family expression was calculated. Same method was used to calculate the usage of Vα gene families.

T Cell Subset Phenotyping

Splenocytes were harvested from hu. Thor mice. 1×10⁶ cells/mL were placed in RPMI-10 and plated in a non-tissue culture treated 6-well plate. Cells were stimulated with both ionomycin (1 μg/mL) and PMA (50 ng/mL) and treated with Golgi block as described in the Human T_{H}1/T_{H}17 Phenotyping Kit (BD BIOSCIENCES). Control wells were left unstimulated and unblocked. Cells were incubated at 37° C. for 5 hours and then collected and pelleted in polypropylene FACS tubes. Cells were then resuspended in cold BD Cytofix buffer and incubated at room temperature. Cells were pelleted and washed in PBE and the resuspended in 1×BD Perm/Wash Buffer prior to incubation at room temperature. After a final centrifugation, cells were stained with the following antibody cocktail: anti-human CD4-PerCP/Cy5.5, IL-17A-PE, IFNγ-FITC, CD45-APC, and anti-mouse-APC/Cy7 (BD BIOSCIENCES). Cells were fixed in 2% paraformaldehyde prior to FACS analysis.

Mixed Lymphocyte Reaction

hu.Thor mice were sacrificed and their spleens and bone marrow were harvested. Splenocytes and bone marrow cells from the same animal were combined, counted, and labeled with carboxyfluorescein succinimidyl ester (CFSE) (INVITROGEN), except for a small portion that was set aside as unlabeled as a negative control. In brief, 1×10⁷-1×10⁸ hu. Thor responder cells were resuspended in a 10 μM working solution of CFSE in PBS. Cells were incubated at 37° C. while kept protected from light and staining was quenched through the addition of RPMI-10 containing 10% FBS. Cells were then pelleted and resuspended in RPMI-10 and then left to incubate for 10 minutes. A small fraction of these labeled cells were then Fc blocked (BD BIOSCIENCES) stained with the following antibodies to act as the positive control: anti-human CD45-APC, CD3-PE, CD4-PerCP/Cy5.5, CD8-BUV395, and Violet LIVE/DEAD (BD BIOSCIENCES). The remaining cells were counted and 1-2×10⁵ of these labeled responder cells were plated in triplicate in a 96-well round-bottomed plate.

Allogeneic stimulator cells were prepared from human umbilical cord blood samples using mitomycin C treatment. In brief, CD34+ cells isolated from umbilical cord blood were cultured and counted. Cell suspensions were made at 5×10⁷ cells/mL in PBS. Mitomycin C (SIGMA-ALDRICH) was added to the cells at 50 μg/mL and the cells were incubated at 37° C. while kept protected from light. The reaction was quenched by adding an excess of complete RPMI-10 media. Cells were then thoroughly washed three times by centrifugation at 300×g for 10 minutes so as to remove all traces of mitomycin-c from the cells to reduce the potential of proliferative responses upon the addition of the stimulator cells to the reaction. Cells were then resuspended in complete RPMI-10 and counted. Cells were plated at 2-5×10⁵ stimulator cells/well depending on the responder cell plating density. Stimulator cells were plated at a ratio of 1:3 (responder:stimulator). The plate was left to incubate for 7 days at 37° C. while kept protected from light. On day 7, the wells were stained with the following antibody panel: anti-human CD45-APC, CD3-PE, CD4-PerCP/Cy5.5, CD8-BUV395, and Violet LIVE/DEAD (BD BIOSCIENCES). Samples were then run on BD Influx FACS system and data was analyzed with FLOWJO 10 software.

Teratoma Analysis

The CC1 iPS line (CW70296CC1) was purchased from CELLULAR DYNAMICS. The Y1 iPS line was established in house from skin fibroblast cells of healthy donors, and has been successfully induced to differentiate into (pro)insulin-producing pancreatic β cell-like cells, thymic epithelial progenitor cells and fibroblast-like cells. Both lines have been maintained in mTeSR plus medium (STEMCELL TECHNOLOGY, 05825). Once confluent, cells were dissociated with RELESR (STEMCELL TECHNOLOGY, 05872) as aggregates. The collected cells were resuspended in culture medium at the concentration of 1×10⁶ per 25 mixed with equal volume of GFR (growth factor reduced) Matrigel (CORNING, 356231, thawed on ice), and were slowly drawn into an insulin syringe for intramuscular injection. 1×10⁶ iPSC aggregates were injected slowly to gastrocnemius muscle of hu.Thor and hu.SRC recipients laying on their back, with CC1 cells on the left side and Y1 on the right side. 26 days after injections, the mice were sacrificed for teratoma excision. Excised teratomas were measured and weighed before proceeding to imaging analysis. Immunocompetent FVB and immunocompromised NSG mice were used as negative and positive controls of teratoma formation, respectively.

Teratoma Histological Analysis

Teratoma tissue was collected for both immunofluorescent (IF) and H&E histological analysis. Tissue being used for IF staining was refrigerated in 4% paraformaldehyde (PFA) (ELECTRON MICROSCOPY SCIENCES, Hatfield, Pa.) for 3 hours, washed with PBS, and placed in 30% sucrose solution at 4° C. for at least 3 days. When ready for cutting, the tissue was briefly washed in PBS and embedded in Tissue Plus Optimal Cutting Temperature Clear embedding medium (FISHER HEALTHCARE, Houston, Tex.) over dry ice and cryosectioned into 8 μm sections using the LEICA CM1950 cryostat (LEICA, Wetzlar, Germany). Once ready to stain, slides were removed from −20° C. and rehydrated with PBS. Tissue was then fixed to the slide using 4% PFA, washed with PBS, and permeabilized with 0.5% TRITON X-100 (SIGMA-ALDRICH, St. Louis, Mo.). Slides were washed again and blocked for 1 hour in 1% bovine serum albumin (SIGMA-ALDRICH, St. Louis, Mo.) in PBS. After blocking, the tissue was washed, and the following primary antibodies (ABCAM, Cambridge, U.K.) were added at 1:100 dilution and left to incubate overnight at 4° C.: human anti-CD3 (ab11089) and anti-HLA-A (ab52922). Primary antibody was aspirated and tissue was washed in PBS before the following secondary antibodies (THERMO FISHER, Waltham, Mass.) were added at 1:1000 dilution for 1 hour incubation at room temperature: ALEXAFLUOR 488 goat anti-rabbit IgG (A11034) and ALEXAFLUOR 555 goat anti-rat IgG (A21434). Secondary antibody was aspirated and tissue was washed in PBS. Nuclei were then stained using ProLong Glass Antifade Mountant with NUCBLUE (LIFE TECHNOLOGIES CORPORATION, Eugene, Oreg.). Slides were imaged using the OLYMPUS FLUOVIEW FV1000 confocal microscope (OLYMPUS, Shinjuku, Tokyo, Japan).

Teratoma tissue being used for H&E staining was collected and placed in 10% neutral buffered formalin (RICHARD-ALLEN SCIENTIFIC, Kalamazoo, Mich.). Tissue was then processed and stained within the Allegheny General Hospital Pathology department. Stained slides were then reviewed by multiple board-certified pathologists for appropriate interpretation and analysis.

Serum Analysis to Detect Immunoglobulin Class Switching

Serum was harvested from hu.Thor mouse blood via either facial vein or cardiac puncture. Antibody Isotyping 7-Plex Human PROCARTAPLEX Panel (INVITROGEN) was used to detect normal class switching functions by testing for IgM, IgG, IgA, and IgE levels within hu.Thor serum. The assay was run according to manufacturer's protocol and was analyzed using the LUMINEX FLEXMAP 3D system (LUMINEX Corporation, Austin, Tex.). Hu.Thor samples were compared to control untreated NSG mice to decrease the occurrence of false positives within the assay.

Statistical Analysis

All values are expressed as the mean±standard deviation unless otherwise specified. Statistical analysis and comparisons were performed with GRAPHPAD PRISM Version 8.0 using an unpaired student's t-test (GRAPHPAD Software), unless specified otherwise. The following are the iterations used for p-value significance: *<0.05, **<0.01, ***<0.001. All experiments were run at a minimum of n=3 to gain statistical significance.

### Example 2 3-D Alginate Microenvironment Promotes the Differentiation of Human Pluripotent Stem Cells into Thymic Epithelial Progenitor Cells

It was shown that encapsulation of hESCs within 3-D alginate capsules promoted propagation of the cells in its pluripotent state as well as efficient differentiation into pancreatic islet-like cells upon stage-wise lineage specific induction (Richardson, T., Kumta, P. N. & Banerjee, I. Alginate encapsulation of human embryonic stem cells to enhance directed differentiation to pancreatic islet-like cells. Tissue Eng Part A 20, 3198-3211, 2014). The synergistic chemical and biophysical induction in the 3-D aggregate culture could significantly enhance the pancreatic islet specification of hESCs as compared to 2-D culture. A similar approach was adopted to promote the differentiation of iPSCs to TEPCs.

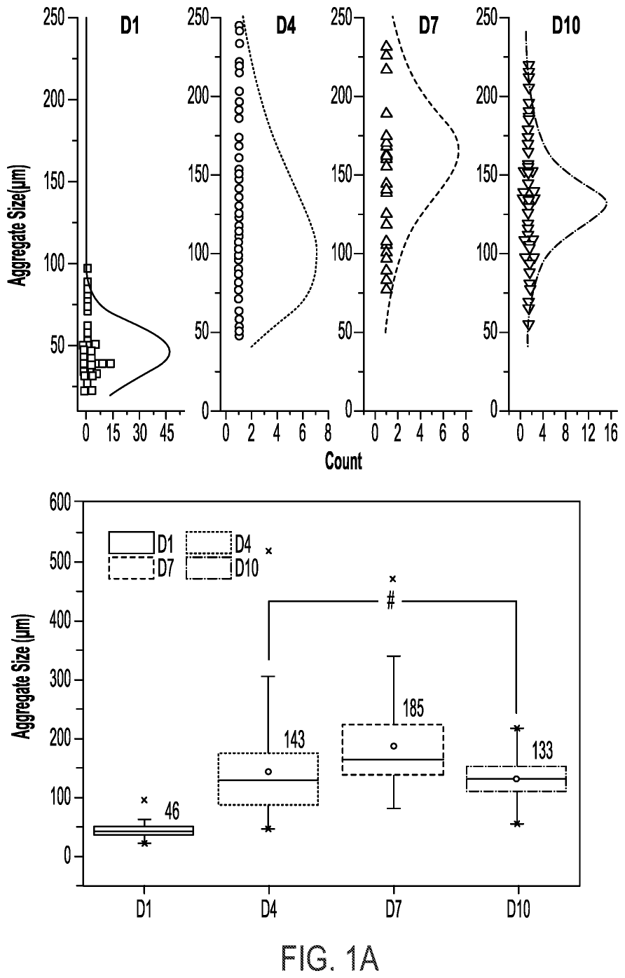

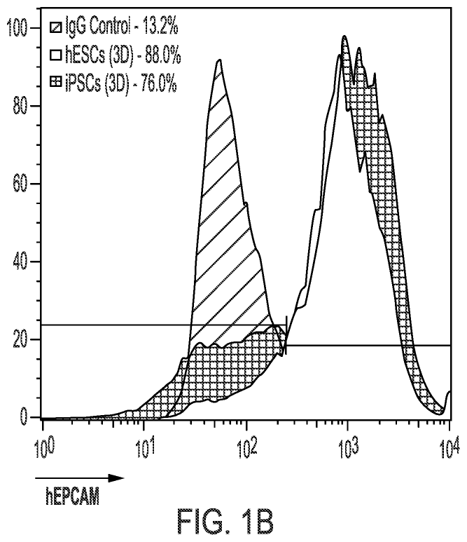

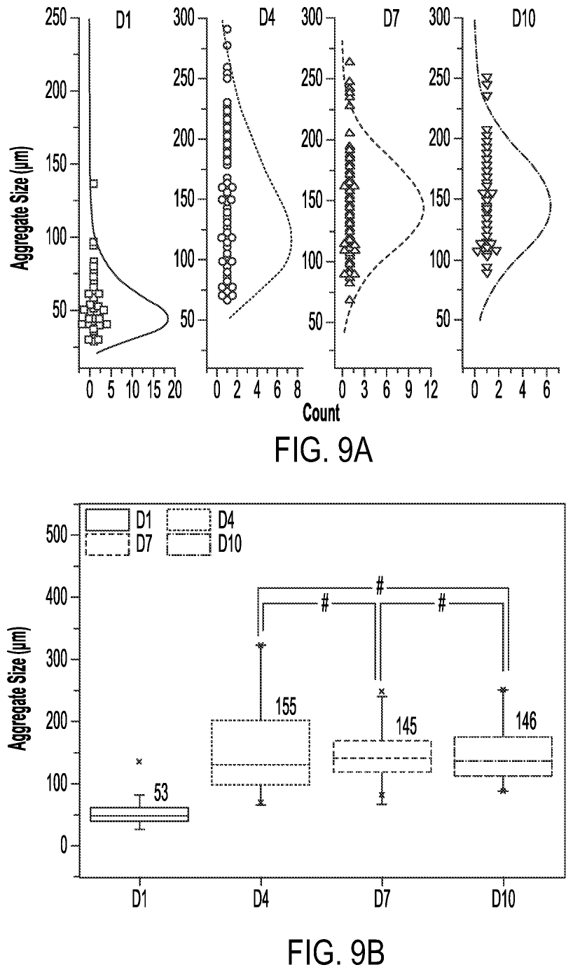

Briefly, iPSCs were embedded in alginate capsules and subjected to a four-stage differentiation protocol, modified from those reported in Parent, A. V. et al. Generation of functional thymic epithelium from human embryonic stem cells that supports host T cell development. Cell stem cell 13, 219-229, doi:10.1016/j.stem.2013.04.004 (2013), Sun, X. et al. Directed differentiation of human embryonic stem cells into thymic epithelial progenitor-like cells reconstitutes the thymic microenvironment in vivo. Cell stem cell 13, 230-236, doi:10.1016/j.stem.2013.06.014 (2013) and Richardson, T., Kumta, P. N. & Banerjee, I. Alginate encapsulation of human embryonic stem cells to enhance directed differentiation to pancreatic islet-like cells. Tissue Eng Part A 20, 3198-3211, 2014. The 3-D environment of the alginate capsules maintained survival of iPSCs encapsulated as single cells, which propagated into small aggregates of approximately 50 um in diameter after 4 to 6 days of culture (FIG. 1a). Encapsulated aggregates were further chemically induced towards definitive endoderm (D1-D4), which resulted in increase of mean aggregate size (box plot, FIG. 1a) and shift in the distribution towards higher aggregate sizes (FIG. 1a, solid lines in the left panel). Further differentiation towards TEPC lineage did not induce further proliferation, as judged from insignificant change in aggregate size, but significantly modified cell phenotype (FIGS. 1a and 1b). The overall efficiency of iPSC to TEPC differentiation was evaluated by examining the surface expression of EpCAM, a key marker for TECs, with flow cytometry (FCM) (FIG. 1b). About 84.6±13% (n=5) of iPSC-derived TEPCs were EpCAM+. Similar patterns of TEPC aggregate formation and differentiation efficiency were observed in inducing hESC differentiation into TEPCs (FIG. 1b and FIG. 9). These results highlight the effectiveness of the 3-D encapsulation to promote the derivation of TEPCs from hPSCs.

To further characterize the iPSC-derived TEPCs, expression of TEC specific markers was analyzed via RT-qPCR analysis. Marked increases in expression of specific cytokeratin markers of thymic epithelium were seen in TEPCs generated in the 3-D alginate capsules when compared to those derived from the 2-D culture. Specifically, CK8 showed a 2-fold increase in expression, while CK17 and CK18 showed an approximate 6-fold and 5-fold increase, respectively. Conversely, both 2-D and 3-D TEPCs displayed significant loss of stem cell (OCT4 and SOX2) and definitive endoderm (SOX17) markers. Additionally, expression of ventral pharyngeal endoderm (VPE) and TEC progenitor markers were significantly increased, further suggesting the successful induction of iPSC differentiation into TEPC lineages. Notably, an approximate 5-fold increase in expression of FOXN1, the master regulator for TEC lineage development, was observed in 3-D TEPCs in comparison to those from 2-D, strongly confirming higher efficiency of TEPC differentiation under the 3-D alginate encapsulation conditions.

Differentiation of epithelial precursors into mature TEC subsets (cTECs and mTECs) is critical for thymic organogenesis and function. To further investigate the progression of TEC differentiation, expression of genes specific to TEC subsets were examined. Genes specific to the cTEC subset that are critical for self-antigen procession and positive selection functions (e.g. PRSS16, ACKR4 and β5t) were expressed at significantly higher levels in 3-D TECs than those of 2-D TECs). Specifically, PRSS16 and ACKR4 showed a 2-fold and a 10-fold increase in expression, respectively. Similarly, the expression of AIRE, a key transcriptional regulator for TSA expression in mTECs that is critical for establishing immunologic self-tolerance, was promoted solely in the 3-D TECs. A similar expression pattern was shared by CSN2, a TSA whose expression in mTECs is regulated by AIRE. Taken together, these data strongly suggest that the 3-D culture configuration is more efficient at promoting TEC lineage differentiation and maturation from iPSCs than the traditional adherent 2-D culture. Similar gene expression patterns were also observed when differentiating hESCs into TEPCs.

### Example 3 Decellularized Thymus Scaffold Microenvironment Supports the Further Differentiation and Maturation of iPSC-Derived TEPCs

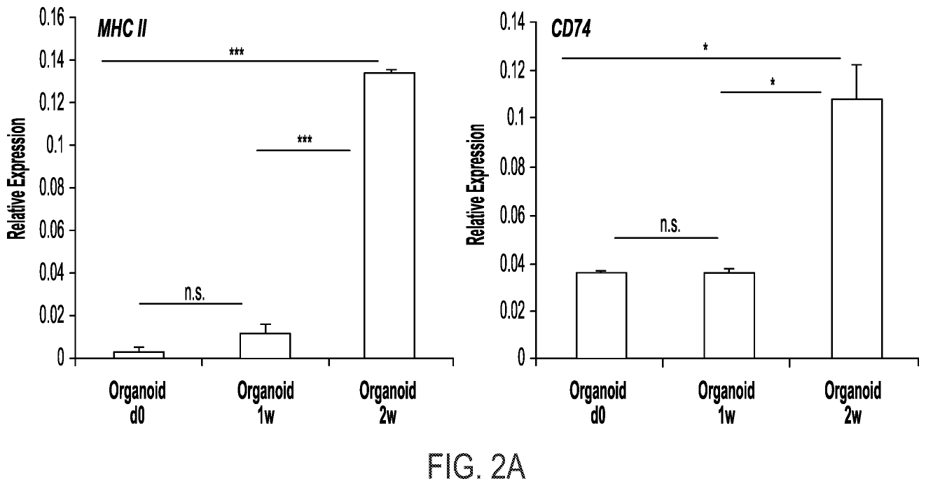

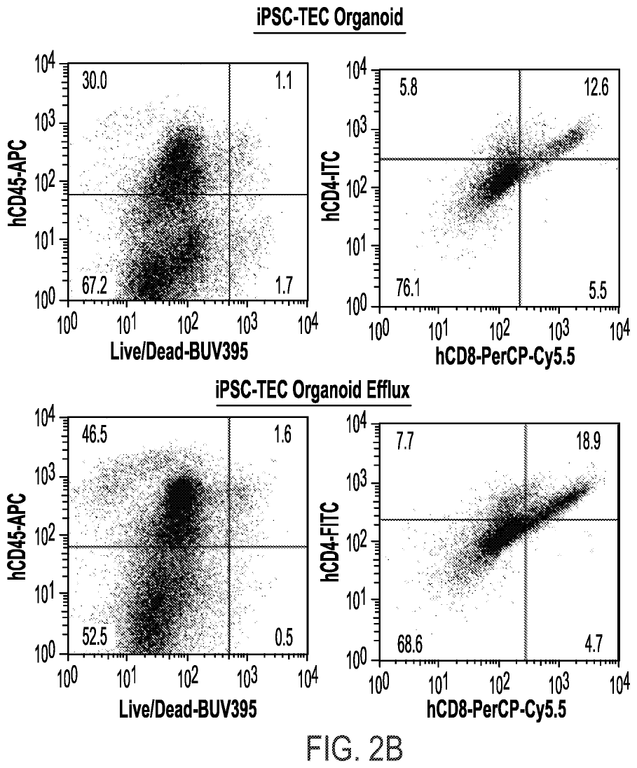

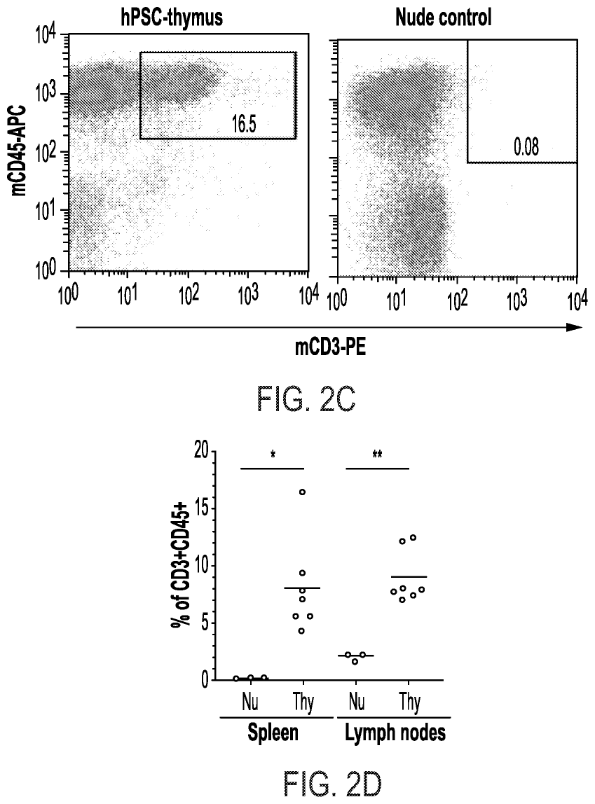

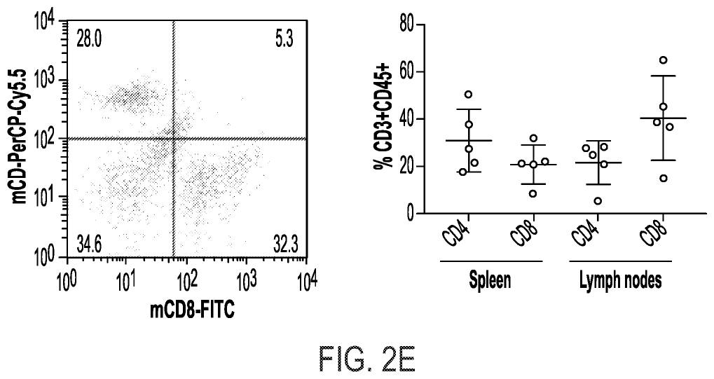

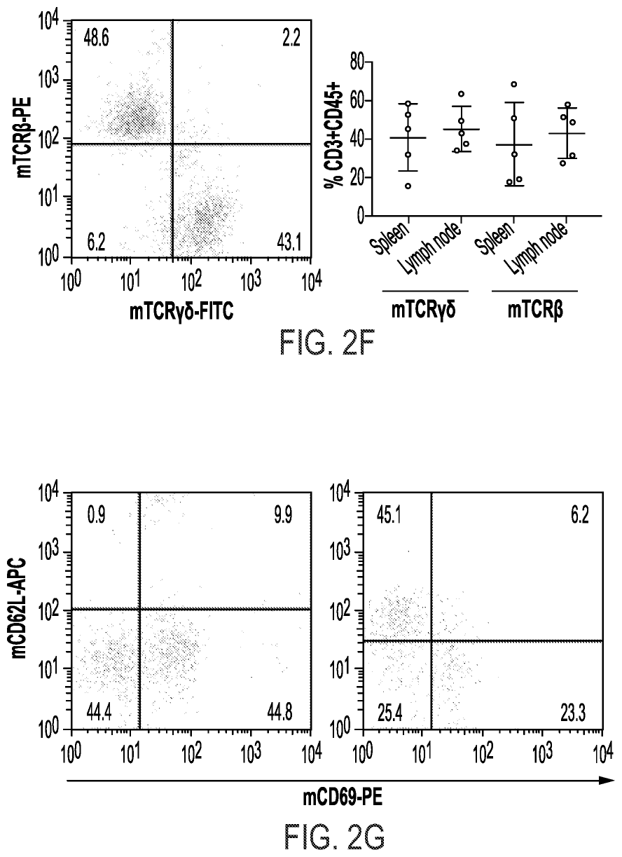

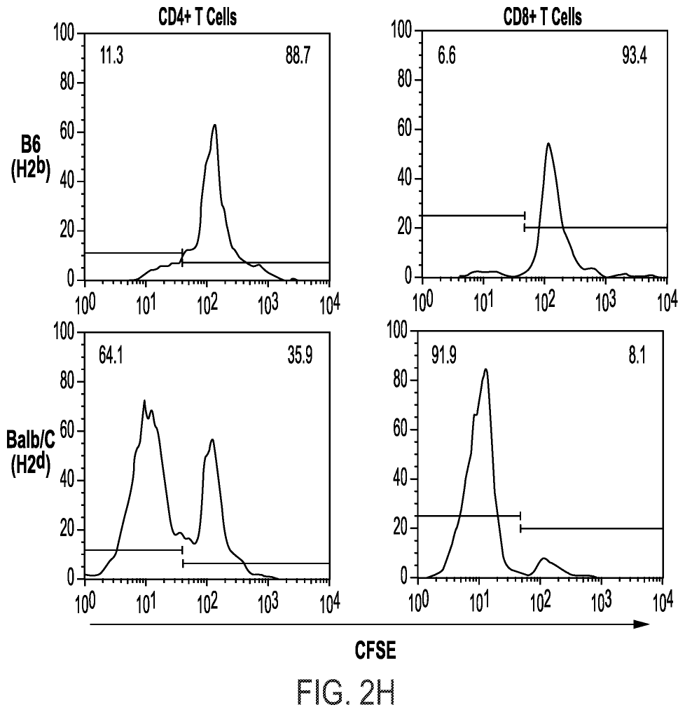

It has been shown that functional thymus organoids can be tissue engineered by repopulating decellularized thymus scaffolds with isolated adult murine TECs. The extracellular matrix (ECM) of the thymus scaffolds can effectively support the survival and the proliferation of adult TECs. To examine whether the thymic ECM can support further the maturation and function of iPSC-derived TECs, TECs were injected into decellularized mouse thymic scaffolds, together with CD34+HSCs isolated from human umbilical cord blood (UCB). The reconstituted human thymus organoids were cultured in the top chambers of transwell culture systems in vitro. Thymic cells within the thymus organoids were able to survive in long-term culture. RT-qPCR analyses showed significant increases in expression of both MHC II and CD74, genes that are essential for TECs to present self-antigens to mediate positive and negative selection of developing T cells (FIG. 2a).

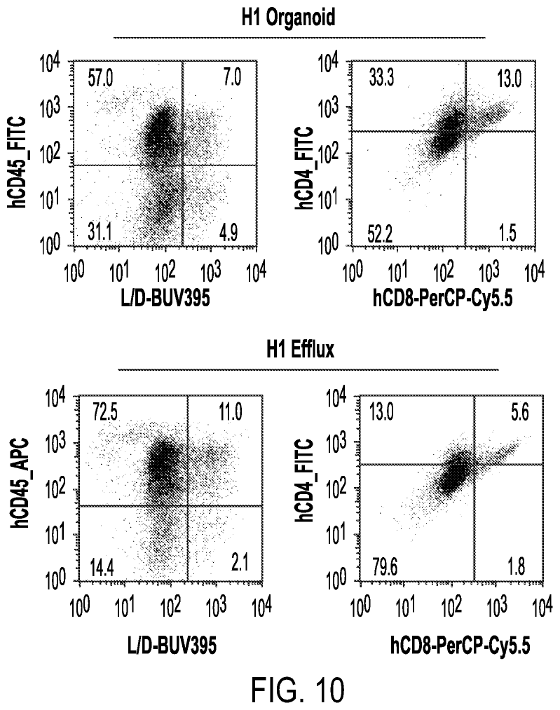

To demonstrate that iPSC-derived TECs can support the de novo generation of human T cells from HSCs, cells were isolated from the human thymus organoids for 21 days of in vitro culture and examined for the surface expression of T cell developing markers with FCM. Thymocytes at various developmental stages, including CD4−CD8− double negative (DN), CD4+CD8+ double positive (DP) and CD4+CD8− or CD4−CD8+ single positive (SP) cells, were detected, suggesting that human thymus organoids tissue-engineered from iPSC-TECs can recapitulate T-lymphopoiesis function in vitro (FIG. 2b and FIG. 10).

To further demonstrate their T-lymphopoiesis function, iPSC-derived TECs (alone, without human HSCs) were injected into decellularized thymus scaffolds and transplanted underneath the kidney capsule of athymic nude mice. iPSC-derived TEC engrafted mice were sacrificed at 18-32 weeks and CD45+CD3+ T cells in the spleens and lymph nodes were further characterized by FCM (FIGS. 2c and 2d). Both CD4+T-helper cells and CD8+ cytotoxic TCRαβ+ T cells were detected, as well as TCRγδ+ T cells (FIGS. 2e and 2f). Similar to previous findings of thymus-engrafted nude mice, majority of the CD8+ T cells displayed the naïve phenotype (CD62L+CD69−), whereas CD4+T helper cells showed the CD62L−CD69+ memory T cell phenotype (FIG. 2g), presumably due to the expansion of CD4+ helper cells under lymphopenic environments. Furthermore, T cells isolated from the thymus organoid-transplanted nude mice underwent robust proliferative responses when challenged with alloantigens in a mixed lymphocyte reaction (MLR) assay, suggesting that iPSC-derived TEC thymus organoids can support the differentiation of endogenous murine bone marrow progenitors to functional mature T cells in vivo (FIG. 2h).

### Example 4 Enhanced Human Hematopoietic Lineage Cell Engraftments in Humanized NSG Mice Transplanted with iPSC-Derived Thymus Organoids

One of the major hurdles in recapitulating human adaptive immune responses in human CD34+HSC-engrafted NSG mice (aka hu.SRC) is the lack of human thymus that can support human T cell development. To overcome this challenge, iPSC-derived thymus organoids were transplanted underneath the kidney capsules of hu.SRC mice to generate the humanized thymus organoid-engrafted mice (hu.Thor). The mice were divided into four groups and received the treatments as shown in the table below:

| | | | hCB | Thymus |
|---|---|---|---|---|
| | Anti-c kit Ab | Busulfan | Transplant | Transplant |
| Group 1 | | + | + | |
| Group 2 | | + | + | + |
| Group 3 | + | + | + | |
| Group 4 | + | + | + | + |

Myeloablative alkylating agent busulfan was used to chemically precondition the NSG recipients prior to HSC and thymus organoid transplantation (Group 2, with Group 1 as control). As it has been shown that treating mice with anti-c-kit antibodies can deplete endogenous murine HSCs in the bone marrow and facilitate donor stem cell engraftment, a c-kit depletion regimen was also evaluated for the generation of hu.Thor mice (Group 4, with Group 3 as control). To ensure that human T cells generated from the iPSC-derived thymus organoids were functionally compatible with the HSC-derived APCs (e.g. dendritic cells, macrophages, and B-cells), only CD34+HSCs isolated from umbilical cord blood (UCB) samples carrying partially matched HLA alleles with the iPSCs were used in the study, unless otherwise stated (Table 3).

TABLE 3
HLA composition of key hPSC cell lines and cord blood samples

| | | | | | | Locus |
|---|---|---|---|---|---|---|
| | | | | Locus | Locus | DQA1, |
| Sample | Locus A | Locus B | Locus C | DRB1 | DRB345 | DQB1 |
| iPS Y1 | A1 A29 | B8 B44 | C7 C16 | DR7 | | DQ2 |
| cells | | | | | | |
| hES H1 | A2 A3 | B8 B35 | C4 C7 | DR1 DR3 | | DQ5 DQ2 |
| cells | | | | | | |
| hCB 8* | A1 A1 | B8 B8 | C7 C7 | DR4 DR17 | DR52 DR53 | DQ2 DQ8 |
| hCB 4 | A1 A2 | B8 B37 | C6 C7 | DR17 DR11 | DR52 DR52 | DQ2 DQ7 |
| hCB 6 | A1 A25 | B8 B44 | C5 C7 | DR4 DR14 | DR52 DR53 | DQ8 DQ5 |
| hCB 7 | A11 A68 | B13 B35 | C10 C4 | DR1 DR16 | DR51 DR51 | DQ5 DQ5 |
| hCB 15 | A1 A30 | B8 B57 | C7 C7 | DR9 DR13 | DR52 DR53 | DQ2 DQ9 |
| hCB 18 | A2 A11 | B18 B27 | C2 C12 | DR1 DR11 | DR52 DR52 | DQ7 DQ5 |

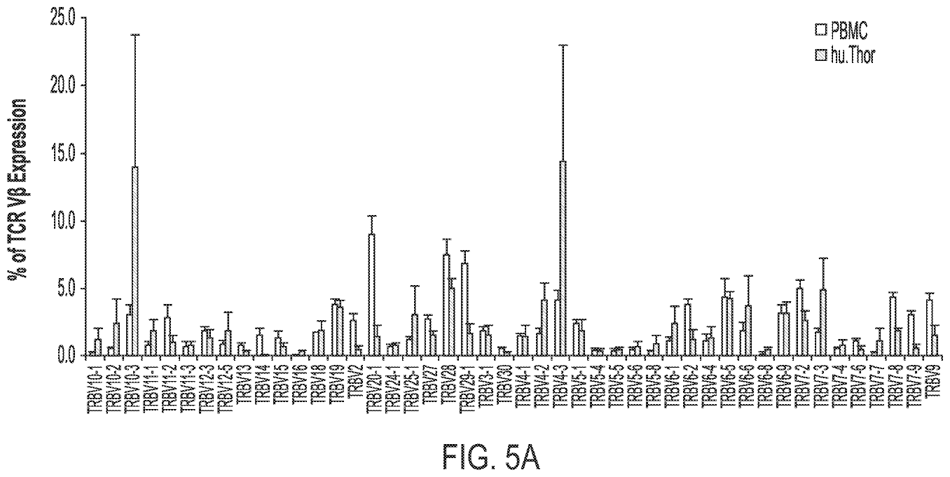

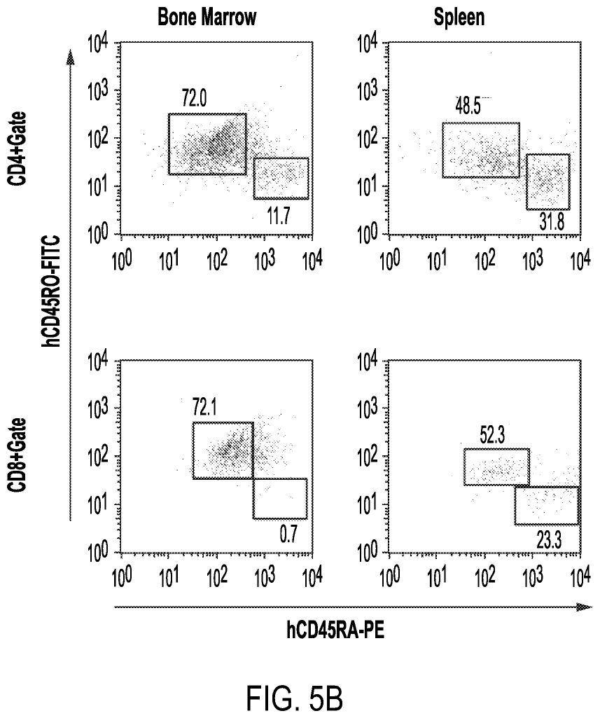

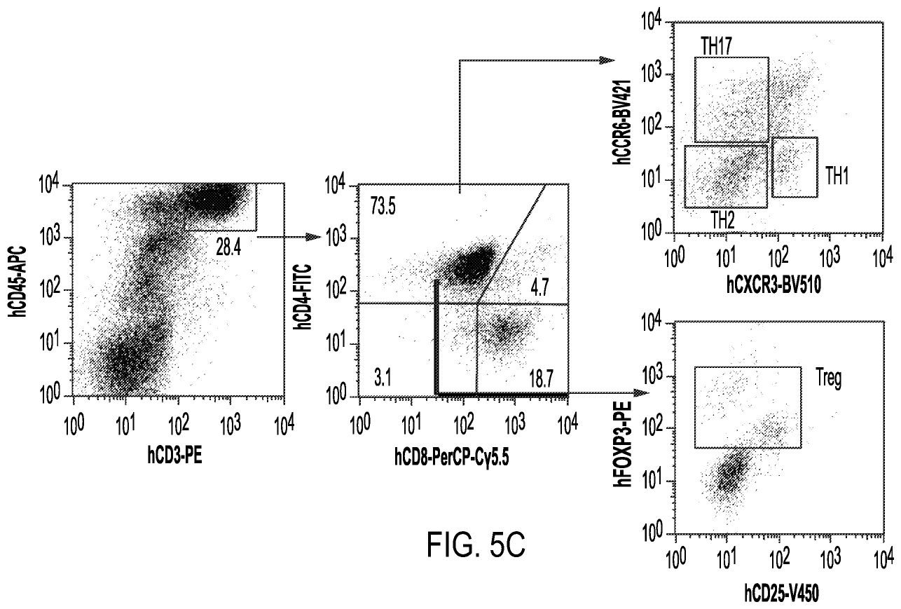

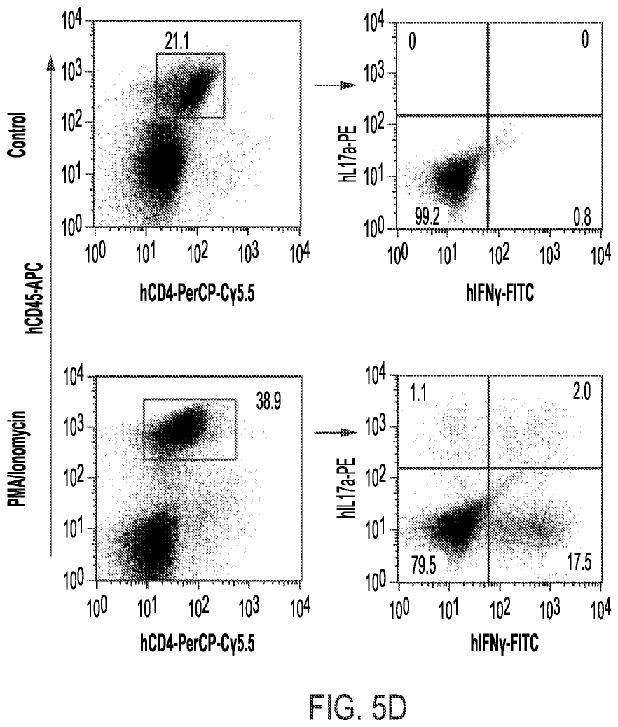

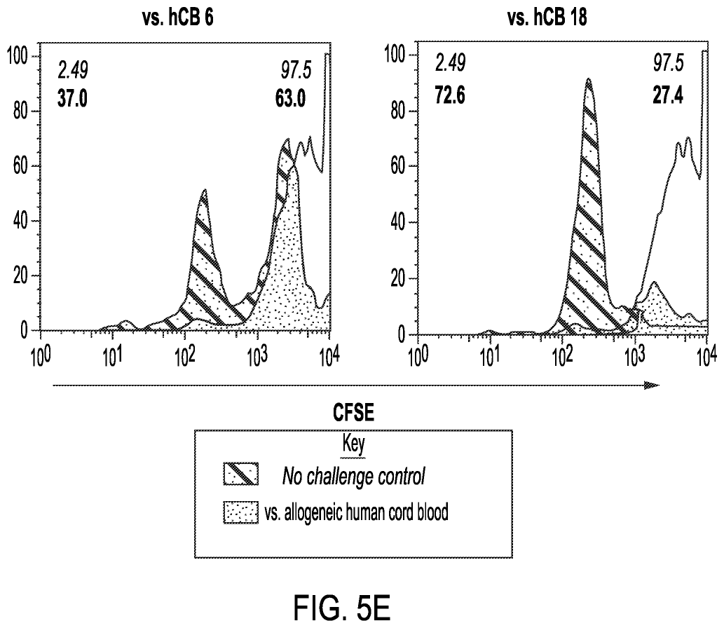

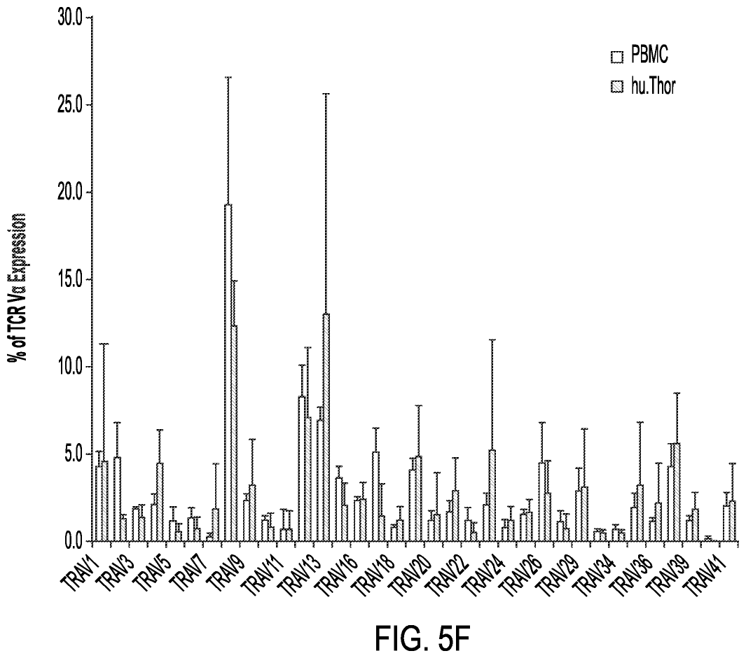

*hCB 8 was used to construct the cohort of mice for the MLR experiments in FIG. 5

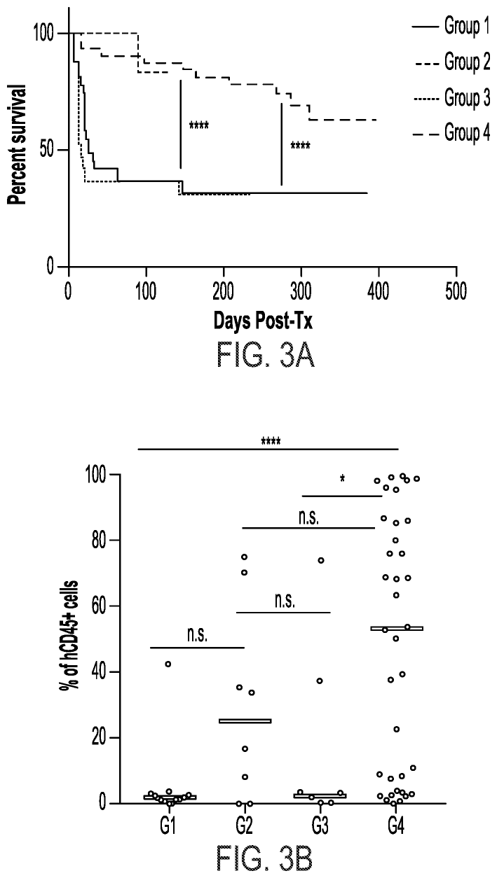

Among the four groups of mice (G1-G4), both control groups (G1 and G3) had significantly worse overall survival than the thymus organoid-engrafted hu. Thor groups (G2 and G4) (FIG. 3a). 56.1% of G1 (n=16/31) and 57.9% G3 (n=11/19) mice died within 30 days after CD34+HSC infusion. Conversely, only three hu.Thor mice (G2, n=0/8; G4, n=3/39) were lost within 30 days post-transplant. These findings suggest that the engrafted human thymus organoids may protect recipients from the detrimental effects associated with the conditioning regimens.

To improve the survival of G1 and G3 mice so that enough controls could be obtained for the study, the cellular engraftment of CD34+HSC was increased five-fold in these mice to approximately 1×10⁶ cells/mouse. Notably, no signs of GVHD, such as decreased body weight, hair loss, or lymphocytic infiltration of organs and tissues, were observed in hu. Thor mice after 350 days in the study, suggesting mutual tolerance between the engrafted hCD34+ human hematopoietic lineage cells and those of the NSG recipients. Even with less numbers of CD34+HSCs transplanted, higher levels of hCD45+ cells were found in the peripheral blood of hu. Thor mice at 12-weeks post transplantation, when compared to controls (FIG. 3b). For example, G4 hu. Thor mice displayed an average of 55% of hCD45+ cells in circulating blood, which was significantly greater than the 4% seen in control G3 mice (FIG. 3b).

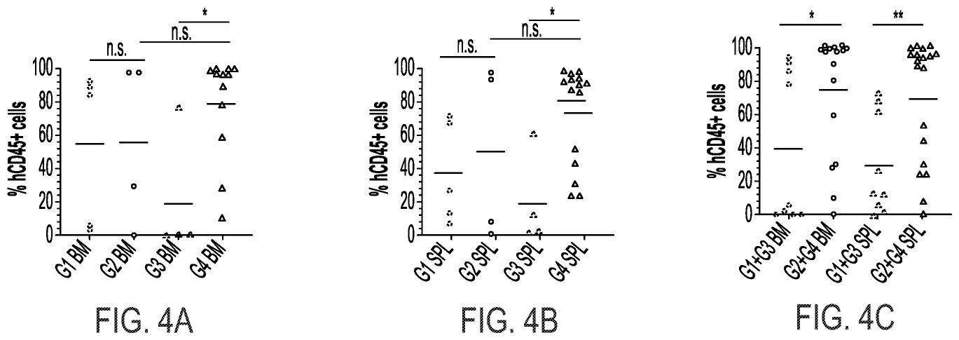

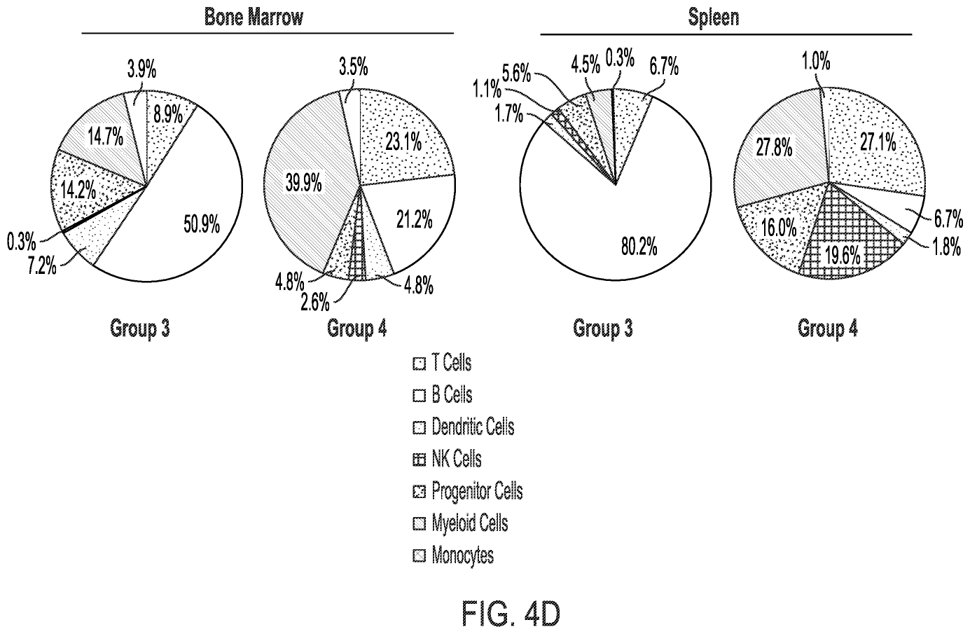

Consistent with the results of peripheral blood analyses, higher levels of human cell chimerism were observed in both the primary (bone marrow) and the secondary (spleen) lymphoid organs in hu. Thor mice (FIG. 4a-b). In particular, G4 mice displayed 80% and 76% of hCD45+ cells in the bone marrow and spleen, respectively, representing a significant increase over those of control G3 mice, which showed only 20% hCD45+ expression in both tissues (FIGS. 4a and 4b). Combined, hu.Thor mice (G2+G4) showed more than 2-fold increases in hCD45+ cells in both bone marrow (2.4-fold) and spleen (2.3-fold) as compared to hu.SRC controls (G1+G3) (FIG. 4c). Further characterization of hu.Thor mice with FCM revealed the development of both lymphoid and myeloid lineage cells in the human hematopoietic compartment (FIG. 4d). Since more robust human cell engraftment and T cell development were observed in G4 mice, efforts were shifted to focus on G4 hu. Thor mice for further characterization of the effects of iPSC-derived thymus organoid transplantation on human T cell development.

### Example 5 iPSC-Derived Thymus Organoids can Support Development of Functional Human T-Helper Cell Subsets in Hu.Thor Mice

A diverse TCR repertoire is critical for an effective adaptive immune response. To assess the overall diversity of T cell populations in hu. Thor mice, expression of Vβ and Vα gene families were examined via a NanoString TCR multiplex assay panel (FIG. 5a and FIG. 5f). Similar levels of reads for each Vβ and Vα family were detected between hu. Thor splenocytes and PBMCs from healthy human donors, demonstrating the complexity of the T cell repertoire in hu.Thor mice. Of note, substantial populations of CD4+T-helper cells and CD8+ cytotoxic T lymphocytes (CTLs) in the spleen displayed the native CD45RA+CD45RO-phenotype (FIG. 5b). Further characterization of T-helper cells showed the development of multiple subsets, including the CXCR3+CCR6−Th1, CXCR3−CCR6+Th17, and CXCR3−CCR6−Th2 cells, as well as CD4+FoxP3+T-regulatory cells (Tregs), the critical population of CD4+ T cells responsible for maintaining immune tolerance (FIG. 5c).

To further demonstrate their functionality of key hu.Thor T cell subsets, hu.Thor T cells were stimulated with phorbol 12-myristate 13-acetate (PMA)/ionomycin, intracellularly stained with anti-IFNγ and anti-IL-17a antibodies and analyzed with FCM. Both IFNγ-producing Th1 and IL-17a-producing Th17 cells were readily detectable, indicating the successful development of multiple functional T-helper lineages in hu.Thor mice (FIG. 5d). Consistently, hu. Thor T cells underwent robust proliferation responses when challenged with allogeneic human cord blood cells that express HLA alleles totally mismatched with those of the engrafted CD34+HSCs and the iPSC-thymus organoids (Table 3) (FIG. 5e). Taken together, these results demonstrate that iPSC-derived thymus organoids can support the development of a diverse and functional repertoire of human T cells in hu. Thor mice.

### Example 6 Hu. Thor Immune Cells Display Similar Gene Expression Profiling as Human PBMCs

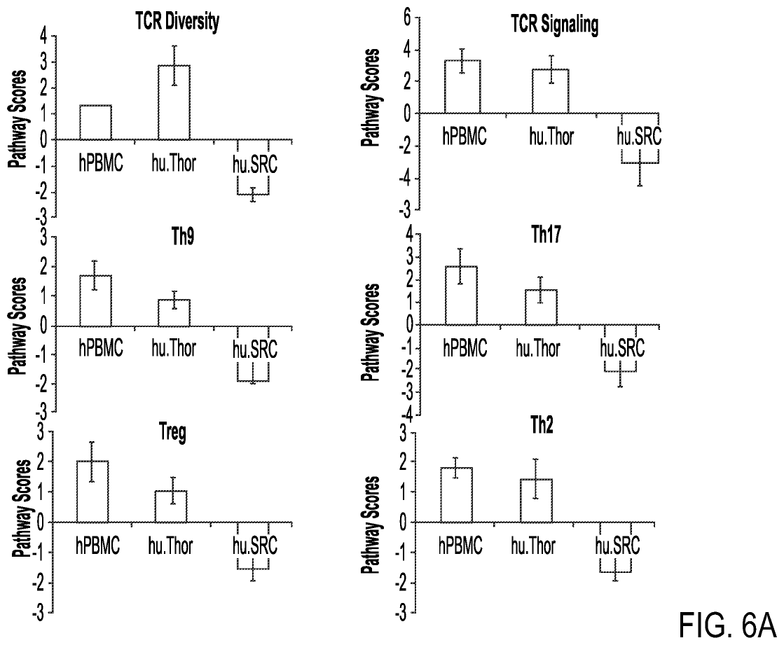

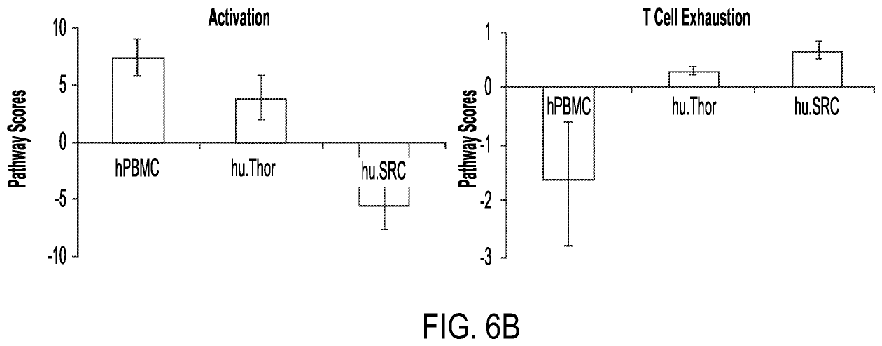

To further characterize the properties of hu.Thor T cells, gene expression profiling analysis was performed on hu. Thor immune cells, focusing on pathways essential to T cell biology, such as T cell diversity, activation, TCR signaling, metabolism and exhaustion. Hu.Thor immune cells exhibited similar overall T cell gene expression profiling as hPBMCs, while differing significantly from hu.SRC cells. Specifically, both hu.Thor and PBMC cells showed higher levels of TCR diversity than hu.SRC cells, suggesting more diverse and complex TCR repertoire (FIG. 6a). Both hu.Thor and PBMC cells also displayed higher TCR signaling phenotypes. Consistently, comparable pathway scores of T-helper subsets, including Th2, Th9, Th17 and Treg cells, were observed between hu.Thor and PBMC cells, notably higher than those of hu.SRC cells (FIG. 6a). Interestingly, higher expression of activation markers were also observed in the hu.Thor and PBMC cells, whereas hu.SRC cells expressed higher T cell exhaustion markers (FIG. 6b). Indeed, higher numbers of transcript reads of T cell exhaustion associated genes (e.g. PDCD1, TNFRSF9/CD137, CD244, HAVCR2/TIM3, and LAGS), as well as lower reads of terminal differentiated memory effector marker KLRG1, were detected in hu.SRC cell. Results from the gene expression profiling and pathway analysis further demonstrated the preeminence of hu. Thor mice over hu.SRC mice to recapitulate the molecular properties of human T cell-mediated immune pathways.

### Example 7 De Novo Generated Human T Cells in Hu.Thor Mice can Effectively Reject Allogeneic Tumor Grafts

It has been shown that neither hu.BLT nor hu.SRC mice are able to effectively reject stem cell-derived allogeneic grafts or teratomas, primarily due to the progressive differentiation of human T cells into the “exhausted” state, marked by increased expression of inhibitory receptors and reduced effector functions (Kooreman, N. G. et al. Alloimmune Responses of Humanized Mice to Human Pluripotent Stem Cell Therapeutics. Cell Reports 20, 1978-1990, 2017). In contrast, hu.Thor T cells mounted effective responses when activated with either antigen-specific (FIG. 5e) or non-specific stimuli (FIG. 5d), and exhibited increased T cell activation pathway profiles (FIG. 6b).

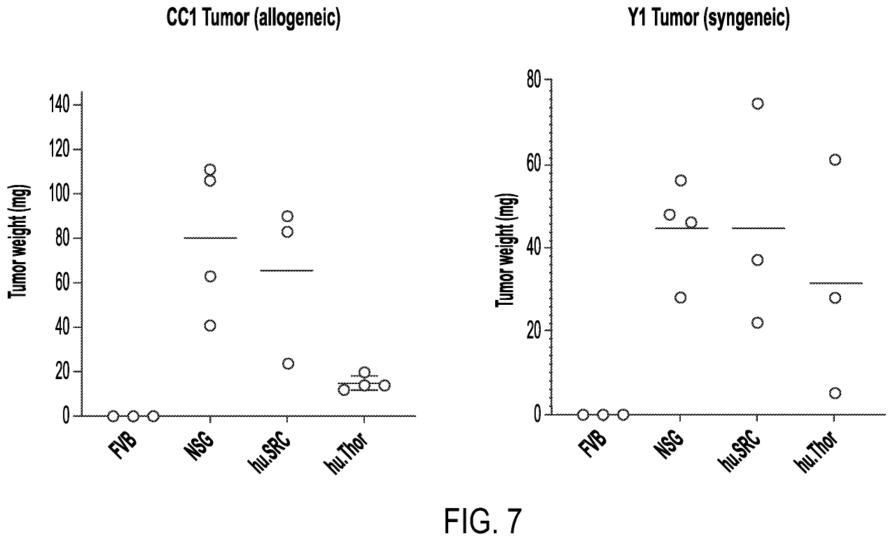

To further challenge the model and test the function of hu.Thor T cells in vivo, teratomas derived from an allogeneic CC1 iPS cell line were generated in hu. Thor mice. Teratomas derived from Y1 iPS cells, with which thymus organoids were generated and engrafted, were used as syngeneic control. Dissociated small clusters of syngeneic Y1 and allogeneic CC1 stem cells were intramuscularly injected to the left and right hind limbs of hu.Thor mice, respectively. Teratomas were harvested at 3-weeks post-inoculation and were measured and weighed (FIG. 7). While robust growth of allogeneic CC1 teratomas was found in NSG and hu.SRC mice, significantly smaller tumor were found in hu.Thor mice (FIG. 7, left panel). Histological examination revealed lymphocytic infiltration of CC1 tumors in both hu.SRC and hu.Thor mice, which is consistent with previous reports. Immunofluorescent analysis of teratoma sections showed increased infiltration of human (HLA-A+) CD3+ T cells in CC1 tumors harvested from the hu. Thor mice. Taken together, these results suggested that hu. Thor T cells can effectively mount alloreactive immune responses to reject allogeneic iPSC-derived tumors, which hu.SRC T cells are incapable of achieving. Of note, no significant difference of syngeneic Y1 tumor growth was observed between NSG, hu.SRC and hu. Thor mice (FIG. 7, right panel), suggesting that Y1 thymus organoid may induce immune tolerance of syngeneic grafts in hu, Thor mice.

### Example 8 De Novo Generated Human T Cells from iPSC-Thymus Organoids can Mediate Humoral Response in Hu. Thor Mice

T cell-dependent activation of B cells plays important roles in both the primary and secondary humoral adaptive immune response. After initial antigen exposure, cytokines secreted by TH2 cells promote the plasma cells to undergo immunoglobulin class switching, shifting from producing IgM to IgG, IgA, or IgE. Recurrent antigen exposure promotes the further maturation of memory B cells to undergo V(D)J somatic hypermutation at the immunoglobulin loci to generate IgGs with higher affinities against the target antigens.

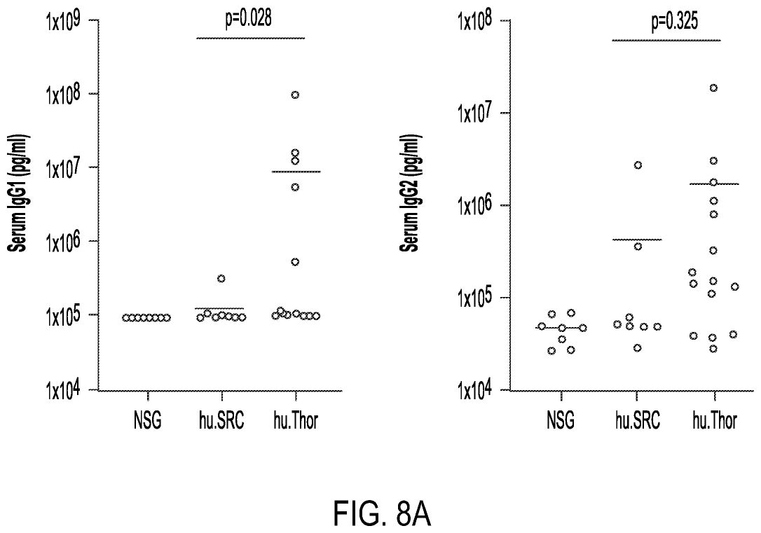

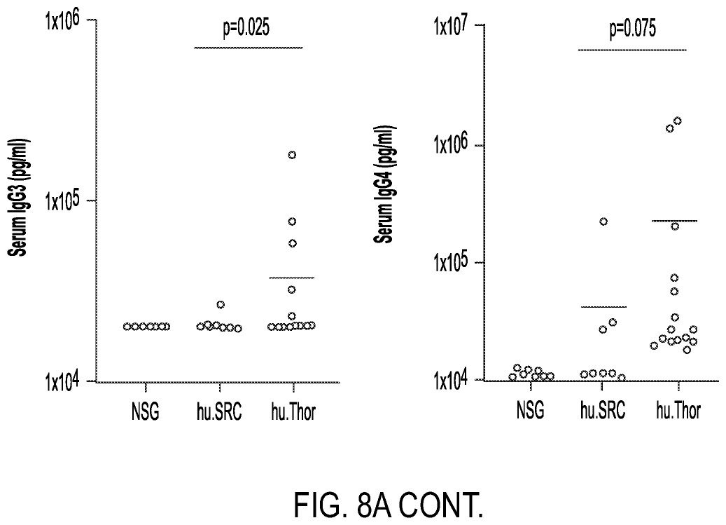

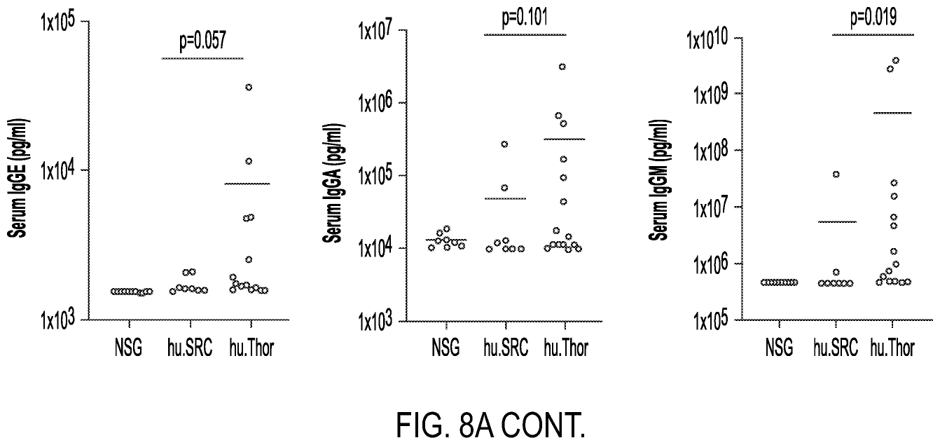

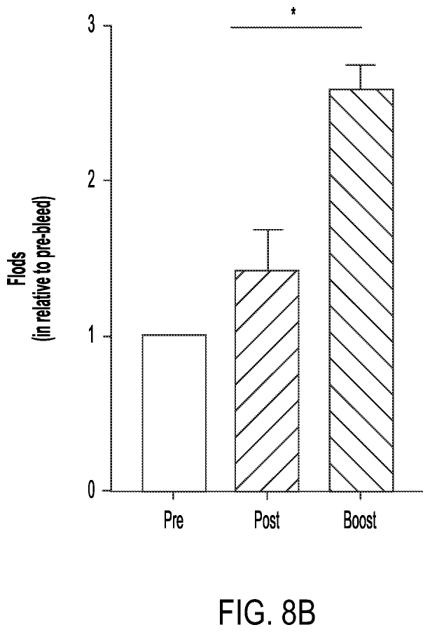

Human antibody isotyping multiplex assays were performed to examine the levels of immunoglobulin classes and subclasses in the sera of hu. Thor mice, in comparison to hu.SRC controls. Major human immunoglobulin classes including IgG, IgM, IgA and IgE, were detected in hu. Thor sera (FIG. 8a). Notably, significant higher levels of IgM and IgG subclasses (IgG1 and IgG3) were observed when compared to hu.SRC samples. These results suggest that human T cells generated from the engrafted iPSC-derived thymus organoids can facilitate B cell maturation and isotype switching function.

To further evaluate their capability to mount effective humoral responses against specific antigens, hu. Thor mice were immunized with vaccines against diphtheria toxoid (DT). IgGs specific to DT were generated after the initial immunization and increased significantly after booster administration (FIG. 8b). These results further prove that T-helper cells generated from iPSC-derived thymus organoids in hu.Thor mice promote the maturation of human B cells and can be used to model humoral responses of the human adaptive immune system.

Hematopoietic humanized mice are powerful small animal models for studying the human immune system. While substantial progress has been made to improve the engraftment and differentiation of multiple lineage immune cells, development of a functional human T cell compartment remains as a major challenge, which significantly hampers the successful modeling of human adaptive immune responses. Over the years, a number of efforts have been made to improve the generation of the human T cells in these mice. Transgenic expression of human SCF, GM-CSF, and IL-3 in NSG-SGM3 mice promotes the stable engraftment of diverse hematopoietic lineages, including CD3+ T cells, CD19+ B cells and CD33+ myeloid cells. The recently generated RG SKI hIL-6 mouse line (Rag2^{-/-}Il2rg^{-/-}SIRPa^{h/m}IL-6^{h/h}), which expresses human IL-6, shows better support for survival of lymphoid lineage cells. These mice develop larger thymus glands and higher numbers of T cells, suggesting that hIL-6 can promote thymopoiesis. Successful IgG class-switch is also observed in antibody-producing B cells, suggesting effective T-helper function. Although transgenic expression of human cytokines/factors can boost the propagation and survival of human T cells, they remain reliant on mouse thymus microenvironments and murine MHCs for T cell education. Human T cells positively selected by mouse TECs will be largely restricted to interact with mouse MHC-expressing APCs, compromising their capability to model human immune responses. While interesting effects have been obtained by transgenically expressing human HLA molecules in mouse cells, the composition of human HLA genes is more complex than that of the mouse (e.g. three MHC I and three MHC II genes in human versus two MHC I and one MHC II genes in mouse). Moreover, formation of functional immunological synapses will depend on the interactions between human costimulatory molecules (e.g. CD28 and CD40) on human T cells and their mouse ligands (e.g. CD80/86 and CD40L) on mouse APCs, further undermining the capability of humanized mice to recapitulate human immune responses. In contrast, iPSC-derived TECs in thymus organoids of hu.Thor mice described herein support the selection of human T cells within a human thymic microenvironment. The data here demonstrate the development and functionality of T helper subsets, such as Th1 and Th17 cells that can produce IFNγ and IL17A, respectively. Low but detectable levels of human interleukin-6 (IL-6) were also found in sera of hu. Thor mice, further suggesting that hu. Thor mice are adept at recapitulating the human immune responses.

The presence of endogenous mouse thymus gland, while degenerated, might still compete with the engrafted thymus organoid for HSC homing. To limit its impact, 8-12 week old NSG mice can be used as recipients, as it has been shown that the hypoplastic thymus glands in NSG mice undergo irreversible, age-associated fibrosis. Indeed, no signs of recovery of endogenous mouse thymus in any hu. Thor mice examined were observed in the study described herein. To model the conditions of patients undergoing chemotherapy, a chemically induced myeloablation regimen, instead of total body lethal irradiation, was used to precondition the NSG mice. Both the age of the recipients and the chemical regimen used in the study could potentially negatively affect the levels of human cell chimerism achieved in the hu.Thor mice. Of interest, higher survival rates were observed in hu.Thor mice compared to hu.SRC controls, suggesting that human cytokines or factors produced from the human thymus organoids may mitigate the chemotoxicity of busulfan.

Although as shown in the examples, HSCs with partially matched HLA can be used as the iPSC-thymus, a fully matched HLA between thymus organoids and transplanted HSCs can better facilitate the recapitulation of human adaptive immunity in hu. Thor mice. Accordingly, humanized mice with both TEPCs and HSCs from a single patient can further improve the modeling of the patient's adaptive immune system. Of note, no clinical signs of GVHD were observed during the life spans of the hu.Thor mice, some of which were kept alive for more than 12 months post-engraftment.

This document discloses here the development of hu.Thor mice capable of generating a vast population of T cell multi-subsets that is able to maintain self-tolerance, as well as mount robust immune responses upon foreign antigen challenge. Human thymus organoids constructed from iPSC lines can support the differentiation of CD34+ human HSCs into functional and diverse CD4+ and CD8+ T cells both in vitro and in vivo. The study highlights the feasibility of recapitulating T-cell mediated human adaptive immune responses from individual patients in small animal models for personalized medicine.

The foregoing examples and description of the preferred embodiments should be taken as illustrating, rather than as limiting this document as defined by the claims. As will be readily appreciated, numerous variations and combinations of the features set forth above can be utilized without departing from this document as set forth in the claims. Such variations are not regarded as a departure from the scope of this document, and all such variations are intended to be included within the scope of the following claims. All references cited herein are incorporated by reference in their entireties.

1. A method for making a bioengineered thymus organoid, comprising
obtaining a cell population comprising human thymic epithelial progenitor cells (TEPCs) or human thymic epithelial cells (TECs) or both;
combining the cell population with human hematopoietic stem cells (HSCs) in a defined ratio to form a combination;
seeding the combination into an extracellular matrix of a de-cellularized thymus scaffold to generate a thymus construct, and
culturing the thymus construct under conditions permitting cellular attachment onto the extracellular matrix thereby making the bioengineered thymus organoid.

2. The method of claim 1, wherein the TEPCs, the TECs, or the HSCs are derived from a donor individual.

3. (canceled)

4. The method of claim 1, wherein the de-cellularized thymus scaffold is from a donor animal.

5. The method of claim 1, wherein the cell population is obtained by a process comprising
encapsulating human pluripotent stem cells (hPSCs) in a suspension medium that separates the hPSCs into single cells;
culturing the hPSCs in a growth medium to increase the number thereof without differentiation;
differentiating the hPSCs to generate TEPCs or TECs in an encapsulation medium, and
freeing the TEPCs or TECs from the encapsulation medium.

6. (canceled)

7. The method of claim 1, wherein the thymus construct is placed into a flow cell with a continuous feed of nutrients and human cells to produce human immune cells.

8. The method of claim 7, wherein the thymus construct comprises immune cells.

9. The method of claim 8, wherein the immune cells comprises B-cells and T-cells.

10. The method of claim 8, wherein the T cells are transduced with a viral vector encoding a chimeric antigen receptor (CAR).

11. (canceled)

12. (canceled)

13. The method of claim 1, wherein the thymus construct is surgically transplanted to a host animal.

14. The method of claim 13, wherein the host animal is a preconditioned humanized immune-deficient animal.

15. The method of claim 14, wherein the host animal is a preconditioned humanized immune-deficient mouse.

16. (canceled)

17. The method of claim 1, wherein the resulting host animal is provided HSCs and produces human immune cells.

18. The method of claim 17, wherein the resulting host animal produces increased quantities of fully human Immunoglobulin G.

19. (canceled)

20. (canceled)

21. (canceled)

22. (canceled)

23. (canceled)

24. (canceled)

25. A bioengineered thymus organoid, comprising
(i) human TEPCs (hTEPCs), human TECs (hTECs), or human HSCs (hHSCs) and
(ii) a thymus scaffold that has been de-cellularized and comprises an extracellular matrix, wherein the hTEPCS, hTECs, or hHSCs attach to the extracellular matrix.

26. (canceled)

27. The bioengineered thymus organoid of claim 25, wherein the bioengineered thymus organoid comprises immune cells.

28. The bioengineered thymus organoid of claim 25, wherein the thymus scaffold is heterologous or allogeneic to the hTEPCs, hTECs, or hHSCs.

29. (canceled)

30. A non-human animal comprising the bioengineered thymus organoid of claim 25.

31. (canceled)

32. (canceled)

33. A method for evaluating of a drug candidate, comprising
(a) contacting a drug candidate with the bioengineered thymus organoid of claim 25; and
(b) detecting the impact of the drug candidate on development of cells that are in the bioengineered thymus organoid or emigrate therefrom.

34. The method of claim 33, wherein the bioengineered thymus organoid is implanted in a host animal and the drug candidate is administered to the host animal.

35. The method of claim 33, wherein the drug candidate is selected from the group consisting of a small molecule, a nucleic acid, a peptide, a polypeptide, an antibody, and an antibody fragment.

36. A method of preparing thymic emigrant cells, comprising
(a) introducing progenitor cells into the bioengineered thymus organoid of claim 25 or a non-human animal comprising the bioengineered thymus organoid;
(b) maintaining the bioengineered thymus organoid or the non-human animal under conditions permitting differentiation of the progenitor cells to generate progeny cells thereof;
(c) egressing the progeny cells from the bioengineered thymus organoid to generate thymic emigrant cells, and
(d) isolating the thymic emigrant cells.

37. Thymic emigrant cells prepared according to the method of claim 36.

38. (canceled)

39. (canceled)

40. A pharmaceutical composition comprising the thymic emigrant cells of claim 37 and a pharmaceutically acceptable carrier.

41. A method for improving the immune function of a subject in need thereof, comprising
administering to the subject an effective amount of the thymic emigrant cells of claim 37.

42. (canceled)

43. A method for improving the immune function of a subject in need thereof, comprising transplanting to the subject the bioengineered thymus organoid of claim 25.
